SEQUENCE LISTING (76 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 4 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 5 - protein, 455 aa, synthetic construct
  REGION 1..455: note = Description of Artificial Sequence: Synthetic polypeptide
MASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRAAAEQKLISEEDLIEFMYPPPYLDNERSNGTIIHIKEKHLCHTQSSPKLFWALVVVAGVLFCYGLLVTVALCVIWTRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 6 - protein, 496 aa, synthetic construct
  REGION 1..496: note = Description of Artificial Sequence: Synthetic polypeptide
MASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRAAAEQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 7 - DNA, 1491 bp, synthetic construct
  misc_feature 1..1491: note = Description of Artificial Sequence: Synthetic polynucleotide
  source 1..1491: mol_type = other DNA
atggcctcaccgttgacccgctttctgtcgctgaacctgctgctgctgggtgagtcgattatcctggggagtggagaagctgaagtccagctgcagcagtctggggctgagcttgtgagacctgggacctctgtgaagttatcttgcaaagtttctggcgataccattacattttactacatgcactttgtgaagcaaaggcctggacagggtctggaatggataggaaggattgatcctgaggatgaaagtactaaatattctgagaagttcaaaaacaaggcgacactcactgcagatacatcttccaacacagcctacctgaagctcagcagcctgacctctgaggacactgcaacctatttttgtatctacggaggatactactttgattactggggccaaggggtcatggtcacagtctcctcaggtggaggtggatcaggtggaggtggatctggtggaggtggatctgacatccagatgacacagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaattgaaacgggcggccgcagaacagaaactgatctctgaagaagacctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 8 - protein, 20 aa, Homo sapiens
MYRMQLLSCIALSLALVTNS

SEQ ID NO: 9 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 10 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 11 - protein, 69 aa, Mus musculus
STTTKPVLRTPSPVHPTGTSQPQRPEDCRPRGSVKGTGLDFACDIYIWAPLAGICVALLLSLIITLICY

SEQ ID NO: 12 - DNA, 207 bp, Mus musculus
  source 1..207: mol_type = other DNA
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 13 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 14 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 15 - DNA, 342 bp, Mus musculus
  source 1..342: mol_type = other DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 16 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 17 - DNA, 123 bp, Mus musculus
  source 1..123: mol_type = other DNA
aatagtagaaggaacagactccttcaaagtgactacatgaacatgactccccggaggcctgggctcactcgaaagccttaccagccctacgcccctgccagagactttgcagcgtaccgcccc

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - DNA, 1368 bp, synthetic construct
  misc_feature 1..1368: note = Description of Artificial Sequence: Synthetic polynucleotide
  source 1..1368: mol_type = other DNA
atggcctcaccgttgacccgctttctgtcgctgaacctgctgctgctgggtgagtcgattatcctggggagtggagaagctgaagtccagctgcagcagtctggggctgagcttgtgagacctgggacctctgtgaagttatcttgcaaagtttctggcgataccattacattttactacatgcactttgtgaagcaaaggcctggacagggtctggaatggataggaaggattgatcctgaggatgaaagtactaaatattctgagaagttcaaaaacaaggcgacactcactgcagatacatcttccaacacagcctacctgaagctcagcagcctgacctctgaggacactgcaacctatttttgtatctacggaggatactactttgattactggggccaaggggtcatggtcacagtctcctcaggtggaggtggatcaggtggaggtggatctggtggaggtggatctgacatccagatgacacagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaattgaaacgggcggccgcagaacagaaactgatctctgaagaagacctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 21 - protein, 158 aa, Mus musculus
SIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 22 - DNA, 81 bp, Homo sapiens
  source 1..81: mol_type = other DNA
ttttgggtgctggtggtggttggtggagtcctggcttgctatagcttgctagtaacagtggcctttattattttctgggtg

SEQ ID NO: 23 - protein, 15 aa, synthetic construct
  REGION 1..15: note = Description of Artificial Sequence: Synthetic peptide
GGGGSGGGGSGGGGS

SEQ ID NO: 24 - protein, 20 aa, Mus musculus
MYSMQLASCVTLTLVLLVNS

SEQ ID NO: 25 - protein, 20 aa, Homo sapiens
METPAQLLFLLLLWLPDTTG

SEQ ID NO: 26 - protein, 20 aa, Mus musculus
METDTLLLWVLLLWVPGSTG

SEQ ID NO: 27 - protein, 21 aa, Homo sapiens
MALPVTALLLPLALLLHAARP

SEQ ID NO: 28 - protein, 16 aa, Homo sapiens
MKWVTFISLLFSSAYS

SEQ ID NO: 29 - protein, 30 aa, Homo sapiens
MDSKGSSQKGSRLLLLLVVSNLLLCQGVVS

SEQ ID NO: 30 - protein, 442 aa, synthetic construct
  REGION 1..442: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEFMYPPPYLDNERSNGTIIHIKEKHLCHTQSSPKLFWALVVVAGVLFCYGLLVTVALCVIWTRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 31 - DNA, 1329 bp, synthetic construct
  misc_feature 1..1329: note = Description of Artificial Sequence: Synthetic polynucleotide
  source 1..1329: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 32 - protein, 444 aa, synthetic construct
  REGION 1..444: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1335 bp, synthetic construct
  misc_feature 1..1335: note = Description of Artificial Sequence: Synthetic polynucleotide
  source 1..1335: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 34 - protein, 483 aa, synthetic construct
  REGION 1..483: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 35 - DNA, 1452 bp, synthetic construct
  misc_feature 1..1452: note = Description of Artificial Sequence: Synthetic polynucleotide
  source 1..1452: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctga

SEQ ID NO: 36 - protein, 485 aa, synthetic construct
  REGION 1..485: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 1458 bp, synthetic construct
  misc_feature 1..1458: note = Description of Artificial Sequence: Synthetic polynucleotide
  source 1..1458: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 38 - protein, 486 aa, synthetic construct
  REGION 1..486: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1461 bp, synthetic construct
  misc_feature 1..1461: note = Description of Artificial Sequence: Synthetic polynucleotide
  source 1..1461: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 40 - protein, 5 aa, synthetic construct
  REGION 1..5: note = Description of Artificial Sequence: Synthetic peptide
FYYMH

SEQ ID NO: 41 - protein, 17 aa, synthetic construct
  REGION 1..17: note = Description of Artificial Sequence: Synthetic peptide
RIDPEDESTKYSEKFKN

SEQ ID NO: 42 - protein, 7 aa, synthetic construct
  REGION 1..7: note = Description of Artificial Sequence: Synthetic peptide
GGYYFDY

SEQ ID NO: 43 - protein, 11 aa, synthetic construct
  REGION 1..11: note = Description of Artificial Sequence: Synthetic peptide
QASEDIYSGLA

SEQ ID NO: 44 - protein, 7 aa, synthetic construct
  REGION 1..7: note = Description of Artificial Sequence: Synthetic peptide
GASDLQD

SEQ ID NO: 45 - protein, 9 aa, synthetic construct
  REGION 1..9: note = Description of Artificial Sequence: Synthetic peptide
QQGLTYPRT

SEQ ID NO: 46 - protein, 116 aa, synthetic construct
  REGION 1..116: note = Description of Artificial Sequence: Synthetic polypeptide
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 47 - protein, 108 aa, synthetic construct
  REGION 1..108: note = Description of Artificial Sequence: Synthetic polypeptide
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 48 - protein, 6 aa, synthetic construct
  REGION 1..6: note = Description of Artificial Sequence: Synthetic peptide
GYAFSS

SEQ ID NO: 49 - protein, 6 aa, synthetic construct
  REGION 1..6: note = Description of Artificial Sequence: Synthetic peptide
YPGDGD

SEQ ID NO: 50 - protein, 9 aa, synthetic construct
  REGION 1..9: note = Description of Artificial Sequence: Synthetic peptide
KTISSVVDF

SEQ ID NO: 51 - protein, 7 aa, synthetic construct
  REGION 1..7: note = Description of Artificial Sequence: Synthetic peptide
NVGTNVA

SEQ ID NO: 52 - protein, 6 aa, synthetic construct
  REGION 1..6: note = Description of Artificial Sequence: Synthetic peptide
SATYRN

SEQ ID NO: 53 - protein, 8 aa, synthetic construct
  REGION 1..8: note = Description of Artificial Sequence: Synthetic peptide
FCQQYNRY

SEQ ID NO: 54 - protein, 122 aa, synthetic construct
  REGION 1..122: note = Description of Artificial Sequence: Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 55 - protein, 108 aa, synthetic construct
  REGION 1..108: note = Description of Artificial Sequence: Synthetic polypeptide
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 56 - protein, 266 aa, synthetic construct
  REGION 1..266: note = Description of Artificial Sequence: Synthetic polypeptide
MASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 57 - DNA, 798 bp, synthetic construct
  misc_feature 1..798: note = Description of Artificial Sequence: Synthetic polynucleotide
  source 1..798: mol_type = other DNA
atggcctcaccgttgacccgctttctgtcgctgaacctgctgctgctgggtgagtcgattatcctggggagtggagaagctgaagtccagctgcagcagtctggggctgagcttgtgagacctgggacctctgtgaagttatcttgcaaagtttctggcgataccattacattttactacatgcactttgtgaagcaaaggcctggacagggtctggaatggataggaaggattgatcctgaggatgaaagtactaaatattctgagaagttcaaaaacaaggcgacactcactgcagatacatcttccaacacagcctacctgaagctcagcagcctgacctctgaggacactgcaacctatttttgtatctacggaggatactactttgattactggggccaaggggtcatggtcacagtctcctcaggtggaggtggatcaggtggaggtggatctggtggaggtggatctgacatccagatgacacagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaattgaaacgg

SEQ ID NO: 58 - protein, 269 aa, Mus musculus
RPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKGSISWTHVHPRRPNVSLLSLSLGGEHPVREMWVWGSLLLLPQATALDEGTYYCLRGNLTIERHVKVIARSAVWLWLLRTGG

SEQ ID NO: 59 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 60 - protein, 263 aa, synthetic construct
  REGION 1..263: note = Description of Artificial Sequence: Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 61 - DNA, 789 bp, synthetic construct
  misc_feature 1..789: note = Description of Artificial Sequence: Synthetic polynucleotide
  source 1..789: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 62 - protein, 18 aa, Homo sapiens
MALPVTALLLPLALLLHA

SEQ ID NO: 63 - protein, 42 aa, Homo sapiens
KRGRKKLLYIFKQPFMRPVQTTQEEDGCSCRFPEEEEGGCEL

SEQ ID NO: 64 - DNA, 126 bp, Homo sapiens
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 65 - protein, 41 aa, Mus musculus
NSRRNRLLQSDYMNMTPRRPGLTRKPYQPYAPARDFAAYRP

SEQ ID NO: 66 - DNA, 123 bp, Mus musculus; identical to SEQ ID NO: 17
  source 1..123: mol_type = other DNA

SEQ ID NO: 67 - protein, 41 aa, Homo sapiens
RSKRSRLLHSDYMNMTPRRPGPTRKHYQPYAPPRDFAAYRS

SEQ ID NO: 68 - DNA, 123 bp, Homo sapiens
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 69 - protein, 113 aa, Mus musculus; identical to SEQ ID NO: 14

SEQ ID NO: 70 - DNA, 339 bp, Mus musculus
  source 1..339: mol_type = other DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgc

SEQ ID NO: 71 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - DNA, 336 bp, Homo sapiens
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 73 - protein, 27 aa, Mus musculus
FWALVVVAGVLFCYGLLVTVALCVIWT

SEQ ID NO: 74 - DNA, 81 bp, Mus musculus
  source 1..81: mol_type = other DNA
ttttgggcactggtcgtggttgctggagtcctgttttgttatggcttgctagtgacagtggctctttgtgttatctggaca

SEQ ID NO: 75 - protein, 27 aa, Homo sapiens
FWVLVVVGGVLACYSLLVTVAFIIFWV

SEQ ID NO: 76 - DNA, 81 bp, Homo sapiens; identical to SEQ ID NO: 22
  source 1..81: mol_type = other DNA

(19) United States
(12) Patent Application Publication
(10) Pub. No.: US 2023/0242879 A1
(43) Pub. Date: Aug. 3, 2023

(54) IL-33 SECRETING IMMUNORESPONSIVE CELLS AND USES THEREOF

(71) Applicant: MEMORIAL SLOAN-KETTERING CANCER CENTER, New York, NY (US)

(72) Inventors: Anthony Daniyan, New York, NY (US); Renier J. Brentjens, New York, NY (US)

(73) Assignee: MEMORIAL SLOAN-KETTERING CANCER CENTER, New York, PA (US)

(21) Appl. No.: 18/188,795

(22) Filed: Mar. 23, 2023

Related U.S. Application Data
(62) Division of application No. 15/930,947, filed on May 13, 2020.
Application No. 15/930,947 is a continuation of application No. PCT/US2018/060992, filed on Nov. 14, 2018.
(60) Provisional application No. 62/585,833, filed on Nov. 14, 2017.

(51) Int. Cl. C12N 5/0783; A61K 38/17; A61K 38/20; C07K 14/47; C12N 15/867; C07K 14/725; C07K 16/28; C07K 14/705; C07K 14/54; A61K 35/17; A61K 39/00

(52) U.S. Cl. CPC C12N 5/0636; A61K 38/177; A61K 38/20; C07K 14/4748; C12N 15/867; C07K 14/7051; C07K 16/2803; C07K 14/70521; C07K 14/54; A61K 35/17; A61K 39/001112; C07K 14/535

(57) ABSTRACT

The present disclosure provides methods and compositions for enhancing the immune response toward cancers and pathogens. It relates to an immunoresponsive cell comprising an antigen-recognizing receptor (e.g., a chimeric antigen receptor (CAR) or a T cell receptor (TCR)), and expressing increased level of IL-33. In certain embodiments, the engineered immunoresponsive cells are antigen-directed and have enhanced immune-activating properties.

### CROSS-REFERENCE TO RELATED APPLICATION

This application is a Divisional Application of U.S. patent application Ser. No. 15/930,947, filed on May 13, 2020, which is a Continuation of International Patent Application No. PCT/US2018/060992, filed on Nov. 14, 2018, which claims priority to U.S. Provisional Application No. 62/585,833 filed on Nov. 14, 2017, the contents of each of which are hereby incorporated by reference in their entireties, and to each of which priority is claimed.

### SEQUENCE LISTING

The specification further incorporates by reference the Sequence Listing submitted herewith on Mar. 23, 2023, which is identified as 0727341439.xml, is 102,400 bytes in size, and was created on Mar. 23, 2023. The Sequence Listing electronically filed herewith, does not extend beyond the scope of the specification and thus does not contain new matter.

### INTRODUCTION

The presently disclosed subject matter provides methods and compositions for enhancing the immune response toward cancers and pathogens. It relates to immunoresponsive cells comprising antigen-recognizing receptors (e.g., chimeric antigen receptors (CARs) or T cell receptors (TCRs)) that are engineered to express an IL-33 polypeptide. These engineered immunoresponsive cells are antigen-directed, promote recruitment of other cytokines and exhibit enhanced anti-target efficacy.

### BACKGROUND OF THE INVENTION

The majority of adult B-cell malignancies, including acute lymphoblastic leukemia (ALL), chronic lymphocytic leukemia, and non-Hodgkin's lymphoma, are incurable despite currently available therapies. Adoptive therapy with genetically engineered autologous T cells has shown evidence of therapeutic efficacy in melanoma and indolent B cell malignancies. T cells may be modified to target tumor-associated antigens through the introduction of genes encoding artificial T-cell receptors, termed chimeric antigen receptors (CAR), specific to such antigens. Immunotherapy is a targeted therapy that has the potential to provide for the treatment of cancer.

However, malignant cells adapt to generate an immunosuppressive microenvironment to protect themselves from immune recognition and elimination. This “hostile” tumor microenvironment poses a challenge to methods of treatment involving stimulation of an immune response, such as targeted T cell therapies. Various modifications have been made toward improving the antitumor effect of CAR- or TCR-engineered T cells. For example, Pegram et al. describes a murine model of CAR-engineered T cells that constitutively secrete interleukin 12 (IL-12) and showed increased cytotoxicity towards CD19⁺ tumor cells (Pegram et al., BLOOD, Vol. 119, No. 18, 2012). However, the secretion of IL-12 led to suppression of interleukin 2 (IL-2), an important cytokine that promotes the proliferation and anti-tumor effect of T and B lymphocytes. Dotti et al. discloses CAR-engineered T cells that constitutively secrete interleukin 15 (IL-15) and an inducible caspase-9 based suicide gene (iC9), which showed increase cytotoxicity towards CD19⁺ tumor cells (US 20130071414 A1). This modified CAR-T cell demonstrated unchanged levels of IL-2 expression both in vivo and in vitro. Accordingly, novel therapeutic strategies for treating neoplasia are urgently required.

### SUMMARY OF THE INVENTION

The presently disclosed subject matter provides immunoresponsive cells (e.g., T cells, Tumor Infiltrating Lymphocytes, Natural Killer (NK) cells, cytotoxic T lymphocytes (CTLs), Natural Killer T (NK-T) cells or regulatory T cells) that (a) express an antigen-recognizing receptor (e.g., CAR or TCR) directed toward a target antigen of interest, and (b) express (and secrete) an interleukin 33 (“IL-33”) polypeptide. In certain non-limiting embodiments, the immunoresponsive cell comprises a nucleotide acid encoding an IL-33 polypeptide (e.g., IL-33 polypeptide-encoding nucleic acid), in expressible form.

The presently disclosed subject matter also provides an immunoresponsive cell comprising (a) an antigen-recognizing receptor (e.g., CAR or TCR) directed toward a target antigen of interest, and (b) a modified promoter at an endogenous (native) IL-33 gene locus, wherein the modified promoter enhances the gene expression of the endogenous IL-33 gene locus. In certain non-limiting embodiments, the modification comprises replacement of an endogenous promoter with a constitutive promoter or an inducible promoter, or insertion of a constitutive promoter or inducible promoter to the promoter region of the endogenous IL-33 gene locus. In certain non-limiting embodiments, the constitutive promoter is selected from the group consisting of a CMV promoter, an EF1a promoter, a SV40 promoter, a PGK1 promoter, a Ubc promoter, a beta-actin promoter, and a CAG promoter. In certain non-limiting embodiments, the inducible promoter is selected from the group consisting of a tetracycline response element (TRE) promoter and an estrogen response element (ERE) promoter.

In certain embodiments, the immunoresponsive cell constitutively expresses the IL-33 polypeptide (mature or non-mature form of IL-33 protein). In certain embodiments, the IL-33 polypeptide is secreted. The antigen-recognizing receptor can be a TCR or a CAR. In certain embodiments, the antigen-recognizing receptor is a CAR. In certain embodiments, the immunoresponsive cell is selected from the group consisting of a T cell, a Natural Killer (NK) cell, a cytotoxic T lymphocyte (CTL), a regulatory T cell, a Natural Killer T (NK-T) cell, a human embryonic stem cell, and a pluripotent stem cell from which lymphoid cells may be differentiated. In certain embodiments, the immunoresponsive cell is autologous.

Furthermore, the presently disclosed subject matter provides methods of using such immunoresponsive cells for inducing and/or enhancing an immune response, and/or for treating and/or preventing a neoplasm (e.g., cancer), infectious disease, and other diseases/disorders that would benefit from an augmented immune response.

In certain non-limiting embodiments, the presently disclosed subject matter provides an isolated immunoresponsive cell (a) comprising an antigen-recognizing receptor that binds to an antigen, and (b) expressing or secreting an IL-33 polypeptide. In certain embodiments, the immunoresponsive cell comprises an exogenous IL-33 polypeptide. In certain embodiments, the immunoresponsive cell comprises a nucleic acid encoding an IL-33 polypeptide. In certain embodiments, binding of the antigen-recognizing receptor to the antigen is capable of activating the immunoresponsive cell. In certain embodiments, the antigen-recognizing receptor is a CAR.

The presently disclosed subject matter further provides a pharmaceutical composition comprising an effective amount of the immunoresponsive cells disclosed herein and a pharmaceutically acceptable excipient. In certain embodiments, the pharmaceutical composition is a pharmaceutical composition for treating and/or preventing a neoplasm (e.g., cancer), wherein the antigen to which the antigen-recognizing receptor binds is a tumor antigen.

The presently disclosed subject matter provides the immunoresponsive cells disclosed herein or the compositions disclosed herein for use in a therapy, e.g., for use in reducing tumor burden, treating and/or preventing a neoplasm, lengthening survival of a subject having a neoplasm, and/or increasing immune-activating cytokine production in response to a tumor antigen or a pathogen antigen in a subject.

The presently disclosed subject matter also provides a method of treating and/or preventing a neoplasm in a subject. In certain embodiments, the method comprises administering to the subject an effective amount of the immunoresponsive cells or the pharmaceutical composition disclosed herein. The presently disclosed subject matter also provides a method of reducing tumor burden in a subject. In certain embodiments, the method comprises administering to the subject an effective amount of the immunoresponsive cells or the pharmaceutical composition disclosed herein. The presently disclosed subject matter further provides a method of lengthening survival of a subject having neoplasm (e.g., cancer). In certain embodiments, the method comprises administering to the subject an effective amount of the immunoresponsive cells or the pharmaceutical composition disclosed herein.

The presently disclosed subject matter also provides a method of enhancing or increasing an immune response to a target antigen in a subject. In certain embodiments, the method comprises administering to the subject an effective amount of the immunoresponsive cells or the pharmaceutical composition disclosed herein. The cell can express and secrete the IL-33 polypeptide that enhances the subject's immune response toward the target antigen.

The presently disclosed subject matter further provides a method of increasing immune-activating cytokine production in response to a cancer or pathogen in a subject. In certain embodiments, the method comprises administering to the subject an effective amount of the immunoresponsive cells or the pharmaceutical composition disclosed herein. In certain non-limiting embodiments, the immune-activating cytokine is selected from the group consisting of IL-2, GM-SCF and IFN-γ. In certain non-limiting embodiments, the immune-activating cytokine is selected from the group consisting of IL-5, IL-9 and IL-13. The presently disclosed subject matter further provides a method of treating blood cancer in a subject in need thereof. In certain embodiments, the method comprises administering to the subject a therapeutically effective amount of the immunoresponsive cells or the pharmaceutical composition disclosed herein. In certain embodiments, the cells are T cells. In certain embodiments, the antigen to which the antigen-recognizing receptor binds is CD19.

The presently disclosed subject matter further provides a method for producing an immunoresponsive cell disclosed herein. In certain embodiments, the method comprises introducing into an immunoresponsive cell (a) a first nucleic acid sequence that encodes an antigen-recognizing receptor that binds to an antigen, and (b) a second nucleic acid sequence that encodes an IL-33 polypeptide.

The presently disclosed subject matter further provides a nucleic acid composition comprising (a) a first nucleic acid sequence encoding an antigen-recognizing receptor (e.g., a CAR or TCR) that binds to an antigen and (b) a second nucleic acid sequence encoding an IL-33 polypeptide (mature or non-mature form of IL-33).

In certain non-limiting embodiments, the first or the second nucleic acid sequence is operably linked to a promoter element constitutively or inducibly expressed in the immunoresponsive cell. The promoter for the first nucleic acid sequence may be the same or different from the promoter for the second nucleic acid sequence. In certain non-limiting embodiments, each of the first and second nucleic acid sequences is operably linked to a promoter element constitutively or inducibly expressed in the immunoresponsive cell. One or both of the first and second nucleic acid sequences may be comprised in a vector, which may be the same vector (bicistronic) or separate vectors. In certain non-limiting embodiments, the vector is a virus vector, e.g., a retroviral vector.

In certain embodiments, the nucleic acid composition is comprised in a vector. In certain non-limiting embodiments, the vector is a virus vector, e.g., a retroviral vector. The presently disclosed subject matter also provides a vector comprising the nucleic acid composition disclosed herein.

The presently disclosed subject matter provides a kit for inducing and/or enhancing an immune response and/or treating and/or preventing a neoplasm (e.g., cancer) or, pathogen infection. In certain embodiments, the kit comprises the immunoresponsive cells disclosed herein, the pharmaceutical composition disclosed herein, the nucleic acid composition disclosed herein, or the vector disclosed herein. In certain embodiments, the kit further comprises written instructions for inducing and/or enhancing an immune response and/or treating and/or preventing a neoplasm or a pathogen infection.

In various non-limiting embodiments, the immunoresponsive cell is autologous to its intended recipient subject.

In various embodiments of any of the aspects delineated herein, the antigen-recognizing receptor is a TCR or a CAR. In various embodiments of any of the aspects delineated herein, the antigen-recognizing receptor is exogenous or endogenous. In various embodiments of any of the aspects delineated herein, the antigen-recognizing receptor is recombinantly expressed. In various embodiments of any of the aspects delineated herein, the antigen-recognizing receptor is expressed from a vector. In various embodiments of any of the aspects delineated herein, the antigen-recognizing receptor is a CAR. In certain embodiments, the CAR comprises an extracellular antigen-binding domain, a transmembrane domain, and an intracellular signaling domain. In certain embodiments, wherein the CAR does not comprise a co-stimulatory signaling domain. In certain embodiments, the CAR is 19z.

In various embodiments of any of the aspects delineated herein, the antigen-recognizing receptor is a TCR. In certain embodiments, the TCR is a recombinant TCR. In certain embodiments, the TCR is a non-naturally occurring TCR. In certain embodiments, the TCR differs from any naturally occurring TCR by at least one amino acid residue. In certain embodiments, the TCR is modified from a naturally occurring TCR by at least one amino acid residue.

In various embodiments of any of the aspects delineated herein, the antigen to which the antigen-recognizing receptor binds is a tumor antigen or a pathogen antigen. In certain embodiments, the antigen is a tumor antigen. In various embodiments of any of the aspects delineated herein, the tumor antigen is selected from the group consisting of CD19, MUC16, MUC1, CAIX, CEA, CD8, CD7, CD10, CD20, CD22, CD30, CD33, CLL1 CD34, CD38, CD41, CD44, CD49f, CD56, CD74, CD133, CD138, a cytomegalovirus (CMV) infected cell antigen, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, Mesothelin, ERBB2, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, and PRAME. In certain embodiments, the antigen is CD19. Amino acid sequences that specifically bind to said antigens are known in the art or may be prepared using methods known in the art; examples include immunoglobulins, variable regions of immunoglobulins (e.g. variable fragment (“Fv”) or bivalent variable fragment (“Fab”)), single chain antibodies, etc. In certain embodiments, the antigen is a pathogen antigen.

In various non-limiting embodiments of any of the aspects delineated herein, the exogenous IL-33 polypeptide is secreted. In various non-limiting embodiments of any of the aspects delineated herein, the IL-33 polypeptide is comprised (and expressed) from a vector. In various non-limiting embodiments of any of the aspects delineated herein, the IL-33 polypeptide comprises a heterologous signal sequence at the amino-terminus (e.g., a signal sequence that is not naturally associated with IL-33). In various embodiments of any of the aspects delineated herein, the heterologous signal sequence is selected from the group consisting of IL-2 signal sequence, the kappa leader sequence, the CD8 leader sequence, and combinations and/or synthetic variations thereof which retain the capacity to promote secretion of IL-33 polypeptide (either mature or non-mature). In certain embodiments, the IL-33 peptide is a mature form of IL-33 protein, or a functional fragment thereof. In certain embodiments, the IL-33 peptide comprises an amino acid sequence that is at least about 80% homologous to the sequence set forth in SEQ ID NO: 4 or SEQ ID NO: 21. In certain embodiments, wherein the IL-33 peptide comprises the amino acid sequence set forth in SEQ ID NO: 4 or SEQ ID NO: 21. In various embodiments of any of the aspects delineated herein, the IL-33 polypeptide enhances an immune response of the immunoresponsive cell. In certain embodiments, the exogenous IL-33 polypeptide increases anti-tumor cytokine production. In certain embodiments, the anti-tumor cytokine is selected from the group consisting of IL-2, GM-CSF and IFN-γ.

In various non-limiting embodiments of any of the aspects delineated herein, the immunoresponsive cell induces prolonged B-cell aplasia compared to an immunoresponsive cell expressing the antigen-recognizing receptor alone (e.g., not comprising an exogenous IL-33 polypeptide). In various non-limiting embodiments of any of the aspects delineated herein, the immunoresponsive cell activates an endogenous immune cell. In certain embodiments, the endogenous immune cell is selected from the group consisting of a NK cell, a NK T cell, a dendritic cell, a macrophage and an endogenous CD8 T cell. In various non-limiting embodiments of any of the aspects delineated herein, the immunoresponsive cell increases the endogenous immune cell population.

In various embodiments of any of the aspects delineated herein, the method reduces the number of tumor cells, reduces tumor size, eradicates the tumor in the subject, reduces the tumor burden in the subject, eradicates the tumor burden in the subject, increases the period of time to relapse/recurrence, and/or increases the period of survival.

Illustrative neoplasms for which the presently disclosed subject matter can be used include, but are not limited to leukemias (e.g., acute leukemia, acute lymphocytic leukemia, acute myelocytic leukemia a, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In various non-limiting embodiments of any of the aspects delineated herein, the neoplasm is one or more of blood cancer, B cell leukemia, multiple myeloma, lymphoblastic leukemia (ALL), chronic lymphocytic leukemia, non-Hodgkin's lymphoma, and ovarian cancer. In certain embodiments, the blood cancer is one or more of B cell leukemia, multiple myeloma, acute lymphoblastic leukemia (ALL), chronic lymphocytic leukemia, and non-Hodgkin's lymphoma. In certain embodiments, the antigen is CD19. In certain embodiments, the neoplasm is ovarian cancer, and the antigen is MUC16.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1C depict various CAR constructs. A) Schematic representation of various CARs, including ah19mZ (a first generation CAR comprising a mouse anti-human CD19 scFv and an intracellular signaling domain that comprises a mouse CD3ζ polypeptide. The amino acid sequence and corresponding nucleotide sequence for ah19mZ are set forth in SEQ ID NOS: 30 and 31, respectively); ah19m28Z (a second generation CAR comprising an antigen-binding domain that is a mouse anti-human CD19 scFv and an intracellular signaling domain that comprises a mouse CD3ζ polypeptide and a costimulatory signaling domain that comprises a mouse CD28 polypeptide. The amino acid sequence and corresponding nucleotide sequence for ah19m28Z are set forth in SEQ ID NOS: 34 and 35, respectively); am19m28Z (a second generation CAR comprising an antigen-binding domain that is a rat anti-mouse CD19 scFv and an intracellular signaling domain that comprises a mouse CD3ζ polypeptide and a costimulatory signaling domain that comprises a mouse CD28 polypeptide. The amino acid sequence and corresponding nucleotide sequence for ah19m28Z are set forth in SEQ ID NOS: 6 and 7, respectively); ah19mZp2A_IL-33 (a first generation CAR (ah19mZ) secreting murine IL-33), ah19m28Zp2A_IL-33 (a second generation CAR (ah19m28Z) secreting murine IL-33), am19m28Zp2A_IL-33 (a second generation CAR (am19m28Z) secreting murine IL-33). All CARs utilized a CD28 proximal extracellular and transmembrane domain as a hinge. In all murine CAR constructs, the cytokine was separated from the CAR by a self-cleaving P2A element. B) first-generation anti-mouse CD19 myc-tag CAR incorporating constitutively-secreted murine IL33. C) first-generation anti-human CD 19 (SJ25C scFv) CAR incorporating constitutively-secreted human IL33. All vectors comprised SFG backbone.

FIG. 2 depicts the cytokine secretion of various modified T cells. CAR T cells were cocultured alone or with antigen-positive tumor cells, +EL4h 19mitoC (EL4h 19 cells were exposed to mitomycin C to prevent proliferation during assay), at an effector:tumor ratio of 10:1. 36 hours later, supernatant was collected and granulocyte macrophage colony-stimulating factor (GM-CSF) and interferon gamma (IFN-gamma) were measured utilizing a bead-based multiplex assay.

FIG. 3 depicts the in vitro cytotoxicity of various modified T cells. CAR T cells were cocultured with antigen-positive tumor cells (EL4h19gfpLUC) at different effector:tumor ratios. Tumor cell lysis was measured by bioluminescence 24 hours later.

FIGS. 4A-4C depict the survival curves of tumor-bearing mice treated with various modified T cells. A) Survival curves of all subjects. EL4h19-bearing C57BL/6 mice were treated with 1-2×10⁵ CAR T cells 1 day post tumor inoculation and survival was followed. Untreated: untreated control group; am19MTm28Z (a second generation CAR comprising an antigen-binding domain that is a rat anti-mouse CD19 scFv and an intracellular signaling domain that comprises a mouse CD3ζ polypeptide and a costimulatory signaling domain that comprises a mouse CD28 polypeptide); am19MTm28ZpIL33mat (a second generation CAR (m19MTm28Z) secreting murine IL-33); ah19mZ (a first generation CAR comprising a mouse anti-human CD19 scFv and an intracellular signaling domain that comprises a mouse CD3ζ polypeptide); ah19m28Z (a second generation CAR comprising an antigen-binding domain that is a mouse anti-human CD19 scFv and an intracellular signaling domain that comprises a mouse CD3 polypeptide and a costimulatory signaling domain that comprises a mouse CD28 polypeptide); ah19mZpIL33mat (a first generation CAR (ah19mZ) secreting murine IL-33; ah19m28ZpIL33 (a second generation CAR (ah19m28Z) secreting murine IL-33. Median survival numbers are shown in the table below. B) Survival curves of ah19m28Z and ah19mZpIL33mat (ah19mZ33). C) Survival curves of ah19m28Z and ah19m28ZpIL33mat (ah19m28Z33).

FIG. 5 depicts the induction of B-cell aplasia by various modified T cells. EL4h19-bearing C57BU6 mice were treated with 2×10⁶ CAR T cells 1 day post tumor inoculation. At day 8, peripheral blood was assessed for B-cells by flow cytometry, and quantified as a percentage of CD45+ cells. Measurements were repeated on day 38 on surviving mice, with age-matched controls.

FIG. 6 depicts the alteration of the peripheral distribution of CD11b⁺ cells by various modified T cells. EL4h 19-bearing C57BU6 mice were treated with 2×10⁶ CAR T cells 1 day post tumor inoculation. At day 8, peripheral blood was assessed for neutrophils (Gr-lhi) and macrophages (F4/80hi) by flow cytometry, and quantified as a percentage of CD11b⁺ cells.

FIGS. 7A-7C depict the serum levels of interferon gamma (A), IL-33 (B) and GM-CSF (C) by various modified T cells. EL4h19-bearing C57BL/6 mice were treated with 2×10⁶ CAR T cells 1 day post tumor inoculation. At day 8, peripheral blood was collected and cytokines quantified utilizing a bead-based multiplex assay.

FIG. 8 depicts the structure of the anti-murine CD19 (am19 derived from 1D3 scFv) CARs: am19mDEL (non-functional CAR); am19mDELp2A_IL-33 (non-functional CAR secreting IL-33); am19mZ (a first generation CAR comprising an antigen-binding domain that is a rat anti-mouse CD19 scFv and an intracellular signaling domain that comprises a mouse CD3ζ polypeptide. The amino acid sequence and corresponding nucleotide sequence for am19mZ are set forth in SEQ ID NOS: 5 and 20, respectively); am19mZp2A_IL-33 (a first generation CAR (am19mZ) secreting murine IL-33); am19m28Z (a second generation CAR comprising an antigen-binding domain that is a rat anti-mouse CD19 scFv and an intracellular signaling domain that comprises a mouse CD3 polypeptide and a costimulatory signaling domain that comprises a mouse CD28 polypeptide. The amino acid sequence and corresponding nucleotide sequence for am19m28Z are set forth in SEQ ID NOS: 6 and 7, respectively); am19m28Zp2A_IL-33 (a second generation CAR(am19m28Z) secreting murine IL-33. All constructs utilized a CD28 proximal extracellular and transmembrane domain as a hinge. In all murine IL-33 secreting constructs, the cytokine was separated from the CAR by a self-cleaving P2A element.

FIG. 9 depicts the IL-33 secretion of various modified T cells. CAR T cells were cocultured alone or with antigen-positive tumor cells, +EL4h19Sm19 (EL4 cells purchased from Sigma, with murine knocked in), at an effector:tumor ratio of 1:1. 24 hours later, supernatant was collected and cytokines were measured utilizing a bead-based multiplex assay.

FIG. 10 depicts the IL-2 secretion of various modified T cells. CAR T cells were cocultured alone or with antigen-positive tumor cells, +EL4h19Sm19 (EL4 cells purchased from Sigma, with murine knocked in), at an effector:tumor ratio of 1:1. 24 hours later, supernatant was collected and cytokines were measured utilizing a bead-based multiplex assay.

FIG. 11 depicts the secretion of GM-CSF and interferon-gamma in various modified T cells. CAR T cells were cocultured alone or with antigen-positive tumor cells, +EL4h19Sm19 (EL4 cells purchased from Sigma, with murine knocked in), at an effector:tumor ratio of 1:1. 24 hours later, supernatant was collected and cytokines were measured utilizing a bead-based multiplex assay.

FIG. 12 depicts the B-cell aplasia after treatment of various modified T cells. Escalating doses (1.25×10⁶ to 20×10⁶ per mouse) of CAR T cells were administered to healthy non-tumor-bearing C57BL/6 mice, and on day 15, peripheral blood was assessed for B-cells by flow cytometry, and quantified as a percentage of CD45+ cells.

FIG. 13 depicts the B-cell recovery post treatment with various modified T cells. Escalating doses (1.25×10⁶ to 20×10⁶ per mouse) of CAR T cells were administered to healthy non-tumor-bearing C57BL/6 mice, and on day 42, peripheral blood was assessed for B-cells by flow cytometry, and quantified as a percentage of CD45+ cells. Mean differences between indicated samples, 95% confidence interval thereof and adjusted P values are shown in the table.

FIG. 14 depicts the survival curves of tumor-bearing mice treated with various modified T cells. EL4Sm19 tumor bearing C57BL/6 mice were treated with 2.5×10⁶ CAR T cells 1 day post tumor inoculation and survival was followed. Untreated: untreated control group; am19MTrn28DEL (CAR without an intracellular signaling domain); am19MTm28DELpIL33mat (a CAR without an intracellular signaling domain secreting murine IL-33); am19MTmZ (comprising a rat anti-mouse CD19 scFv and an intracellular signaling domain that comprises a mouse CD3 polypeptide); am19MTmZp33mat (a first generation CAR (am19MTmZ) secreting murine IL-33); am19MTm28Z (M1928Z) (a second generation CAR comprising an antigen-binding domain that is a rat anti-mouse CD19 scFv and an intracellular signaling domain that comprises a mouse CD3 polypeptide and a costimulatory signaling domain that comprises a mouse CD28 polypeptide); am19MTm28Zp33mat (a second generation CAR (am19MTm28Z) secreting murine IL-33). Median survival numbers are shown in the table below.

FIG. 15 depicts the structure of anti-human CD19 CARs: ah19hDEL (nonfunctional CAR containing a truncated CD28 intracellular domain, lacking signaling motifs); ah19hZ (a first generation CAR comprising an antigen-binding domain that is a mouse anti-human CD19 scFv and an intracellular signaling domain that comprises a human CD3ζ polypeptide. The amino acid sequence and corresponding nucleotide sequence for ah19hZ are set forth in SEQ ID NOS: 32 and 33, respectively)); ah19hBBZ (a second generation CAR comprising an antigen-binding domain that is a mouse anti-human CD19 scFv and an intracellular signaling domain that comprises a human CD3ζ polypeptide and a costimulatory signaling domain that comprises a human 4-1BB polypeptide. The amino acid sequence and corresponding nucleotide sequence for ah19hBBZ are set forth in SEQ ID NOS: 38 and 39, respectively); ah19h28Z (a second generation CAR comprising an antigen-binding domain that is a mouse anti-human CD19 scFv and an intracellular signaling domain that comprises a human CD3ζ polypeptide and a costimulatory signaling domain that comprises a human CD28 polypeptide. The amino acid sequence and corresponding nucleotide sequence for ah19h28Z are set forth in SEQ ID NOS:36 and 37, respectively); ah19hDELp2A_IL-33 (non-functional CAR secreting human IL-33); ah19hZp2A_IL-33 (a first generation CAR (ah19hZ) secreting human IL-33); ah19hBBZp2A_IL-33 (a second generation CAR (ah19hBBZ) secreting human IL-33); ah19h28Zp2A_IL-33 (a second generation CAR (ah19h28Z) secreting human IL-33). All constructs utilized a CD28 proximal extracellular and transmembrane domain as a hinge (including 4-1BB-based constructs). In all human IL-33 secreting constructs, the cytokine was separated from the CAR by a self-cleaving P2A element.

FIG. 16 depicts the flow cytometry analyses of cell surface expression of various CAR constructs. GALV-pseudotyped 293GPG packaging cells stably transduced with human-based constructs were assessed for the presence of CAR through the use of an Alexa-647 conjugated anti-idiotype antibody specific to the mouse-derived anti-human CD19 CAR. A no transduced RD114, a similar 293HEK cell, was used as a negative control.

FIG. 17 depicts the IL-33 secretion of packaging cells transduced with various CAR constructs. Supernatant was collected from packaging cells post retroviral transduction and assessed for cytokine concentrations by bead-based multiplex assay.

FIG. 18 depicts the flow cytometry analyses of cell surface expression of various CAR constructs. Human T cells stably transduced with human-based constructs were assessed for the presence of the CAR 5 days post inoculation. Non-transduced human T cells (hTcemp) served as a negative control.

FIGS. 19A-19C depict the cell lysis by the modified T cells. CAR T cells were cocultured with antigen-positive tumor cells: A) DOHH2, B) NALM6, and C) Raji, at different effector:tumor ratios, and tumor cell lysis was measured by bioluminescence 24 hours later.

FIG. 20 is a schematic representation of a construct in accordance with certain embodiments of the presently disclosed subject matter.

FIG. 21 depicts that secretable IL-33 is functional in mouse IL33-secreting CAR T cells. IL33-secreting CAR T cells demonstrated increased antigen-independent interferon gamma secretion on exposure to recombinant murine IL-12 (long/mL).

FIG. 22 depicts that secretable IL-33 is functional in human IL33-secreting CAR T cells. Non-functional CD19-targeted IL-33-secreting CAR T cells with a truncated EGFR (Et.ah19hDELp33) demonstrated increased antigen-independent interferon gamma secretion on exposure to escalating doses of recombinant human IL-12 as compared to equivalent CAR construct not secreting IL-33 (Et.ah19hDEL).

FIG. 23 depicts that secretable IL-33 is functional in human IL-33-secreting first-generation CAR T cells. First-generation human CD19-targeted IL-33-secreting CAR T cells with a truncated EGFR (Et.ah19hZp33) demonstrated increased antigen-independent interferon gamma secretion on exposure to escalating doses of recombinant human IL-12 as compared to equivalent CAR construct not secreting IL-33 (Et.ah19hZ).

FIG. 24 depicts that secretable IL-33 is functional in human IL-33-secreting second-generation CAR T cells. Second-generation human CD19-targeted IL-33-secreting CAR T cells with a truncated EGFR (Et.ah19h28Zp33) demonstrated increased antigen-independent interferon gamma secretion on exposure to escalating doses of recombinant human IL-12 as compared to equivalent CAR construct not secreting IL-33 (Et.ah19h28Z).

### DETAILED DESCRIPTION OF THE INVENTION

The presently disclosed subject matter provides cells, including genetically modified immunoresponsive cells (e.g., T cells, NK cells, or CTL cells) comprising a combination of an antigen-recognizing receptor (e.g., TCR or CAR) and a secretable IL-33 polypeptide (e.g., an exogenous IL-33 polypeptide, or a nucleic acid encoding an IL-33 polypeptide). The presently disclosed subject matter also provides methods of using such cells for inducing and/or enhancing an immune response to a target antigen, and/or treating and/or preventing neoplasia or other diseases/disorders where an increase in an antigen-specific immune response is desired. The presently disclosed subject matter is based, at least in part, on the discovery that a secretable IL-33 polypeptide enhances the anti-tumor effect of an immunoresponsive cell comprising an antigen-recognizing receptor (e.g., a CAR-expressing T cell or a TCR-expressing T cell). It was observed that the co-expression of an IL-33 polypeptide and an antigen-recognizing receptor (e.g., a CAR, such as 19z CAR) on T cells led to increased cytokine secretion.

Malignant cells have developed a series of mechanisms to protect themselves from immune recognition and elimination. The presently disclosed subject matter provides immunogenicity within the tumor microenvironment for tumor eradication, and represents a significant advance over conventional adoptive T cell therapy. The presently disclosed subject matter provides an option of foregoing some or all ancillary treatments such as prior conditioning of the host with total body irradiation, high-dose chemotherapy, and/or post-infusion cytokine support.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF—KB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest (e.g., an IL-33 polypeptide) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, e.g., less than about 500 mM NaCl and 50 mM trisodium citrate, and e.g., less than about 250 mM NaCl and 25 mM trisodium citrate. Low stringency hybridization can be obtained in the absence of organic solvent, e.g., formamide, while high stringency hybridization can be obtained in the presence of at least about 35% formamide, and e.g., at least about 50% formamide. Stringent temperature conditions will ordinarily include temperatures of at least about 30° C., e.g., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., e.g., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasia, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “immunoresponsive cell” is meant a cell that functions in an immune response or a progenitor, or progeny thereof.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in GGGGSGGGGSGGGGS [SEQ ID NO: 23].

By “neoplasm” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [ SEQ ID NO: 8] (human), MYSMQLASCVTLTLVLLVNS [SEQ ID NO: 24] (mouse); the kappa leader sequence: METPAQLLFLLLLWLPDTTG [SEQ ID NO: 25] (human), METDTLLLWVLLLWVPGSTG [SEQ ID NO: 26] (mouse); the CD8 leader sequence: MALPVTALLLPLALLLHAARP [SEQ ID NO: 27] (human); the truncated human CD8 signal peptide: MALPVTALLLPLALLLHA [SEQ ID NO: 62] (human); the albumin signal sequence: MKWVTFISLLFSSAYS [SEQ ID NO: 28] (human); and the prolactin signal sequence: MDSKGSSQKGSRLLLLLVVSNLLLCQGVVS [SEQ ID NO: 29] (human).

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 2. Antigen-Recognizing Receptors

The present disclosure provides antigen-recognizing receptors that bind to an antigen of interest. In certain embodiments, the antigen-recognizing receptor is a chimeric antigen receptor (CAR). In certain embodiments, the antigen-recognizing receptor is a T-cell receptor (TCR). The antigen-recognizing receptor can bind to a tumor antigen or a pathogen antigen.

### 2.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME and ERBB.

In certain embodiments, the antigen-recognizing receptor binds to CD19. In certain embodiments, the antigen-recognizing receptor binds to a murine CD19 polypeptide. In certain embodiments, the murine CD19 polypeptide comprises the amino acid sequence set forth in SEQ ID NO: 58.

[SEQ ID NO: 58]
RPQKSLLVEVEEGGNVVLPCLPDSSPVSSEKLAWYRGNQSTPFLELSPGS
PGLGLHVGSLGILLVIVNVSDHMGGFYLCQKRPPFKDIWQPAWTVNVEDS
GEMFRWNASDVRDLDCDLRNRSSGSHRSTSGSQLYVWAKDHPKVWGTKPV
CAPRGSSLNQSLINQDLTVAPGSTLWLSCGVPPVPVAKGSISWTHVHPRR
PNVSLLSLSLGGEHPVREMWVWGSLLLLPQATALDEGTYYCLRGNLTIER
HVKVIARSAVWLWLLRTGG

In certain embodiments, the antigen-recognizing receptor binds to a humane CD19 polypeptide. In certain embodiments, the human CD19 polypeptide comprises the amino acid sequence set forth in SEQ ID NO: 59.

[SEQ ID NO: 59]
PEEPLVVKVEEGDNAVLQCLKGTSDGPTQQLTWSRESPLKPFLKLSLGLP
GLGIHMRPLAIWLFIFNVSQQMGGFYLCQPGPPSEKAWQPGWTVNVEGSG
ELFRWNVSDLGGLGCGLKNRSSEGPSSPSGKLMSPKLYVWAKDRPEIWEG
EPPCLPPRDSLNQSLSQDLTMAPGSTLWLSCGVPPDSVSRGPLSWTHVHP
KGPKSLLSLELKDDRPARDMWVMETGLLLPRATAQDAGKYYCHRGNLTMS
FHLEITARPVLWHWLLRTGGWK

In certain embodiments, the antigen-recognizing receptor binds to the extracellular domain of a human or murine CD19 protein.

In certain embodiments, the antigen-recognizing receptor binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3t chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3). In certain embodiments, the antigen-recognizing receptor is a first generation CAR. In certain embodiments, the antigen-recognizing receptor is a second generation CAR.

In certain non-limiting embodiments, the extracellular antigen-binding domain of the CAR (embodied, for example, an scFv or an analog thereof) binds to an antigen with a dissociation constant (K_{d}) of about 2×10′ M or less. In certain embodiments, the K_{d} is about 2×10′ M or less, about 1×10′ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain non-limiting embodiments, the K_{d} is about 3×10⁻⁹M or less. In certain non-limiting embodiments, the K_{d} is from about 1×10⁻⁹ M to about 3×10′ M. In certain non-limiting embodiments, the K_{d} is from about 1.5×10⁻⁹ M to about 3×10′ M. In certain non-limiting embodiments, the K_{d} is from about 1.5×10⁻⁹M to about 2.7×10⁻⁷M.

Binding of the extracellular antigen-binding domain (for example, in an scFv or an analog thereof) can be confirmed by, for example, enzyme-linked immunosorbent assay (ELISA), radioimmunoassay (MA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

In accordance with the presently disclosed subject matter, a CARs can comprise an extracellular antigen-binding domain, a transmembrane domain and an intracellular signaling domain, wherein the extracellular antigen-binding domain specifically binds to an antigen, e.g., a tumor antigen or a pathogen antigen.

2.3.1. Extracellular Antigen Binding Domain of A CAR

In certain embodiments, the extracellular antigen-binding domain specifically binds to an antigen. In certain embodiments, the extracellular antigen-binding domain is an scFv. In certain embodiments, the scFv is a human scFv, a humanized scFv, or a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

In certain embodiments, the extracellular antigen-binding domain of a presently disclosed CAR is a murine scFv. In certain embodiments, the extracellular antigen-binding domain of a presently disclosed CAR is a murine scFv that binds to a murine CD19 polypeptide. In certain embodiments, the extracellular antigen-binding domain is a murine scFv, which comprises the amino acid sequence of SEQ ID NO: 56 and specifically binds to a murine CD19 polypeptide (e.g., a murine CD19 polypeptide comprising the amino acid sequence set forth in SEQ ID NO: 58). In certain embodiments, the nucleotide sequence encoding the amino acid sequence of SEQ ID NO: 56 is set forth in SEQ ID NO: 57. In certain embodiments, the murine scFv comprises a heavy chain variable region (V_{H}) comprising the amino acid sequence set forth in SEQ ID NO: 46. In certain embodiments, the murine scFV comprises a light chain variable region (V_{L}) comprising the amino acid sequence set forth in SEQ ID NO: 47. In certain embodiments, the murine scFv comprises a V_{H} comprising the amino acid sequence set forth in SEQ ID NO: 46 and a V_{L} comprising the amino acid sequence set forth in SEQ ID NO: 47, optionally with (iii) a linker sequence, for example a linker peptide, between the V_{H} and the V_{L}. In certain embodiments, the linker comprises amino acids having the sequence set forth in SEQ ID NO: 23. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 46. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 46. In certain embodiments, the extracellular antigen-binding domain comprises a V_{H} comprising the amino acid sequence set forth in SEQ ID NO: 46. In certain embodiments, the extracellular antigen-binding domain comprises a V_{L} comprising an amino acid sequence that is at least about 80% (e.g., at least about 85%, at least about 90%, or at least about 95%) homologous to SEQ ID NO: 47. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 47. In certain embodiments, the extracellular antigen-binding domain comprises a V_{L} comprising the amino acid sequence set forth in SEQ ID NO: 47. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 46, and a V_{L} comprising an amino acid sequence that is at least about 80% (e.g., at least about 85%, at least about 90%, or at least about 95%) homologous to SEQ ID NO: 47. In certain embodiments, the extracellular antigen-binding domain comprises a V_{H} comprising the amino acid sequence set forth in SEQ ID NO: 46 and a V_{L} comprising the amino acid sequence set forth in SEQ ID NO: 47. In certain embodiments, the extracellular antigen-binding domain comprises a V_{H} CDR1 comprising the amino acid sequence set forth in SEQ ID NO: 40, or a conservative modification thereof, a V_{H} CDR2 comprising the amino acid sequence set forth in SEQ ID NO: 41 or a conservative modification thereof, and a V_{H} CDR3 comprising the amino acid sequence set forth in SEQ ID NO: 42, a conservative modification thereof. In certain embodiments, the extracellular antigen-binding domain comprises a V_{H} CDR1 comprising the amino acid sequence set forth in SEQ ID NO: 40, a V_{H} CDR2 comprising the amino acid sequence set forth in SEQ ID NO: 41, and a V_{H} CDR3 comprising the amino acid sequence set forth in SEQ ID NO: 42. In certain embodiments, the extracellular antigen-binding domain comprises a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof. In certain embodiments, the extracellular antigen-binding domain comprises a V_{L} CDR1 comprising the amino acid sequence set forth in SEQ ID NO: 43, a V_{L} CDR2 comprising the amino acid sequence set forth in SEQ ID NO: 44, and a V_{L} CDR3 comprising the amino acid sequence set forth in SEQ ID NO: 45. In certain embodiments, the extracellular antigen-binding domain comprises a V_{H} CDR1 comprising the amino acid sequence set forth in SEQ ID NO: 40 or a conservative modification thereof, a V_{H} CDR2 comprising the amino acid sequence set forth in SEQ ID NO: 41 or a conservative modification thereof, a V_{H} CDR3 comprising the amino acid sequence set forth in SEQ ID NO: 42, a conservative modification thereof, a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof. In certain embodiments, the extracellular antigen-binding domain comprises a V_{H} CDR1 comprising amino acids having the sequence set forth in SEQ ID NO: 40, a V_{H} CDR2 comprising the amino acid sequence set forth in SEQ ID NO: 41, a V_{H} CDR3 comprising the amino acid sequence set forth in SEQ ID NO: 42, a V_{L} CDR1 comprising the amino acid sequence set forth in SEQ ID NO: 43, a V_{L} CDR2 comprising the amino acid sequence set forth in SEQ ID NO: 44, and a V_{L} CDR3 comprising the amino acid sequence set forth in SEQ ID NO: 45.

In certain embodiments, the extracellular antigen-binding domain of a presently disclosed CAR is a murine scFv that binds to a human CD19 polypeptide. In certain embodiments, the extracellular antigen-binding domain is a murine scFv, which comprises the amino acid sequence of SEQ ID NO: 60 and specifically binds to a human CD19 polypeptide (e.g., a human CD19 polypeptide comprising the amino acid sequence set forth in SEQ ID NO: 59). In certain embodiments, the nucleotide sequence encoding the amino acid sequence of SEQ ID NO: 60 is set forth in SEQ ID NO: 61. In certain embodiments, the murine scFv comprises a heavy chain variable region (V_{H}) comprising the amino acid sequence set forth in SEQ ID NO: 54. In certain embodiments, the murine scFV comprises a light chain variable region (V_{L}) comprising the amino acid sequence set forth in SEQ ID NO: 55. In certain embodiments, the murine scFV comprises V_{H} comprising the amino acid sequence set forth in SEQ ID NO: 54 and a V_{L} comprising the amino acid sequence set forth in SEQ ID NO: 55, optionally with (iii) a linker sequence, for example a linker peptide, between the V_{H} and the V_{L}. In certain embodiments, the linker comprises amino acids having the sequence set forth in SEQ ID NO: 23. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 54. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 54. In certain embodiments, the extracellular antigen-binding domain comprises a V_{H} comprising the amino sequence set forth in SEQ ID NO: 54. In certain embodiments, the extracellular antigen-binding domain comprises a V_{L} comprising an amino acid sequence that is at least about 80% (e.g., at least about 85%, at least about 90%, or at least about 95%) homologous to SEQ ID NO: 55. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 55. In certain embodiments, the extracellular antigen-binding domain comprises a V_{L} comprising the amino acid sequence set forth in SEQ ID NO: 55. certain embodiments, the extracellular antigen-binding domain comprises a V_{H} comprising an amino acid sequence that is at least about 80% (e.g., at least about 85%, at least about 90%, or at least about 95%) homologous to SEQ ID NO: 54, and a V_{L} comprising an amino acid sequence that is at least about 80% (e.g., at least about 85%, at least about 90%, or at least about 95%) homologous to SEQ ID NO: 55. In certain embodiments, the extracellular antigen-binding domain comprises a V_{H} comprising the amino acid sequence set forth in SEQ ID NO: 54 and a V_{L} comprising the amino acid sequence set forth in SEQ ID NO: 55. In certain embodiments, the extracellular antigen-binding domain comprises a V_{H} CDR1 comprising the amino acid sequence set forth in SEQ ID NO: 48, or a conservative modification thereof, a V_{H} CDR2 comprising the amino acid sequence set forth in SEQ ID NO: 49 or a conservative modification thereof, and a V_{H} CDR3 comprising the amino acid sequence set forth in SEQ ID NO: 50, a conservative modification thereof. In certain embodiments, the extracellular antigen-binding domain comprises a V_{H} CDR1 comprising the amino acid sequence set forth in SEQ ID NO: 48, a V_{H} CDR2 comprising the amino acid sequence set forth in SEQ ID NO: 49, and a V_{H} CDR3 comprising the amino acid sequence set forth in SEQ ID NO: 50. In certain embodiments, the extracellular antigen-binding domain comprises a V_{L} CDR1 comprising the amino acid sequence set forth in SEQ ID NO: 51 or a conservative modification thereof, a V_{L} CDR2 comprising the amino acid sequence set forth in SEQ ID NO: 52 or a conservative modification thereof, and a V_{L} CDR3 comprising the amino acid sequence set forth in SEQ ID NO: 53 or a conservative modification thereof. In certain embodiments, the extracellular antigen-binding domain comprises a V_{L} CDR1 comprising the amino acid sequence set forth in SEQ ID NO: 51, a V_{L} CDR2 comprising the amino acid sequence set forth in SEQ ID NO: 52, and a V_{L} CDR3 comprising the amino acid sequence set forth in SEQ ID NO: 53. In certain embodiments, the extracellular antigen-binding domain comprises a V_{H} CDR1 comprising the amino acid sequence set forth in SEQ ID NO: 48 or a conservative modification thereof, a V_{H} CDR2 comprising the amino acid sequence set forth in SEQ ID NO: 49 or a conservative modification thereof, a V_{H} CDR3 comprising the amino acid sequence set forth in SEQ ID NO: 50, a conservative modification thereof, a V_{L} CDR1 comprising the amino acid sequence set forth in SEQ ID NO: 51 or a conservative modification thereof, a V_{L} CDR2 comprising the amino acid sequence set forth in SEQ ID NO: 52 or a conservative modification thereof, and a V_{L} CDR3 comprising the amino acid sequence set forth in SEQ ID NO: 53 or a conservative modification thereof. In certain embodiments, the extracellular antigen-binding domain comprises a V_{H} CDR1 comprising amino acids having the sequence set forth in SEQ ID NO: 48, a V_{H} CDR2 comprising the amino acid sequence set forth in SEQ ID NO: 49, a V_{H} CDR3 comprising the amino acid sequence set forth in SEQ ID NO: 50, a V_{L} CDR1 comprising the amino acid sequence set forth in SEQ ID NO: 51, a V_{L} CDR2 comprising the amino acid sequence set forth in SEQ ID NO: 52 and a V_{L} CDR3 comprising the amino acid sequence set forth in SEQ ID NO: 53.

TABLE 1

| | | anti-mouse CD19 scFv (1D3) | | |
|---|---|---|---|---|
| CDRs | | 1 | 2 | 3 |
| V_{H} | a.a. | FYYMH [SEQ ID NO: | RIDPEDESTK YSEKFKN | GGYYFDY [SEQ ID |
| | | 40] | [SEQ ID NO: 41] | NO: 42] |
| V_{L} | a.a. | QASEDIYSGL A [SEQ | GASDLQD [SEQ ID | QQGLTYPRT [SEQ |
| | | ID NO: 43] | NO: 44] | ID NO: 45] |

| | |
|---|---|
| Full V_{H} | EVQLQQSGAE LVRPGTSVKL SCKVSGDTIT FYYMHFVKQR PGQGLEWIGR |
| | IDPEDESTKY SEKFKNKATL TADTSSNTAY LKLSSLTSED TATYFCIYGG |
| | YYFDYWGQGV MVTVSS [SEQ ID NO: 46] |
| Full V_{L} | DIQMTQSPAS LSTSLGETVT IQCQASEDIY SGLAWYQQKP GKSPQLLIYG |
| | ASDLQDGVPS RFSGSGSGTQ YSLKITSMQT EDEGVYFCQQ GLTYPRTFGG |
| | GTKLELKR [SEQ ID NO: 47] |
| scFv | MASPLTRFLS LNLLLLGESI ILGSGEAEVQ LQQSGAELVR PGTSVKLSCK |
| | VSGDTITFYY MHFVKQRPGQ GLEWIGRIDP EDESTKYSEK FKNKATLTAD |
| | TSSNTAYLKL SSLTSEDTAT YFCIYGGYYF DYWGQGVMVT VSSGGGGSGG |
| | GGSGGGGSDI QMTQSPASLS TSLGETVTIQ CQASEDIYSG LAWYQQKPGK |
| | SPQLLIYGAS DLQDGVPSRF SGSGSGTQYS LKITSMQTED EGVYFCQQGL |
| | TYPRTFGGGT KLELKR [SEQ ID NO: 56] |
| DNA | ATGGCCTCACCGTTGACCCGCTTTCTGTCGCTGAACCTGCTGCTGCTGGGTGAGTCG |
| | ATTATCCTGGGGAGTGGAGAAGCTGAAGTCCAGCTGCAGCAGTCTGGGGCTGAGCTT |
| | GTGAGACCTGGGACCTCTGTGAAGTTATCTTGCAAAGTTTCTGGCGATACCATTACA |
| | TTTTACTACATGCACTTTGTGAAGCAAAGGCCTGGACAGGGTCTGGAATGGATAGGA |
| | AGGATTGATCCTGAGGATGAAAGTACTAAATATTCTGAGAAGTTCAAAAACAAGGCG |
| | ACACTCACTGCAGATACATCTTCCAACACAGCCTACCTGAAGCTCAGCAGCCTGACC |
| | TCTGAGGACACTGCAACCTATTTTTGTATCTACGGAGGATACTACTTTGATTACTGG |
| | GGCCAAGGGGTCATGGTCACAGTCTCCTCAGGTGGAGGTGGATCAGGTGGAGGTGGA |
| | TCTGGTGGAGGTGGATCTGACATCCAGATGACACAGTCTCCAGCTTCCCTGTCTACA |
| | TCTCTGGGAGAAACTGTCACCATCCAATGTCAAGCAAGTGAGGACATTTACAGTGGT |
| | TTAGCGTGGTATCAGCAGAAGCCAGGGAAATCTCCTCAGCTCCTGATCTATGGTGCA |
| | AGTGACTTACAAGACGGCGTCCCATCACGATTCAGTGGCAGTGGATCTGGCACACAG |
| | TATTCTCTCAAGATCACCAGCATGCAAACTGAAGATGAAGGGGTTTATTTCTGTCAA |
| | CAGGGTTTAACGTATCCTCGGACGTTCGGTGGCGGCACCAAGCTGGAATTGAAACGG |
| | [SEQ ID NO: 57] |

TABLE 2

| | | anti-human CD19 scFv (SJ25C1) | | |
|---|---|---|---|---|
| CDRs | | 1 | 2 | 3 |
| V_{H} | a.a. | GYAFSS [SEQ ID NO: | YPGDGD [SEQ ID NO: | KTISSVVDF [SEQ |
| | | 48] | 49] | ID NO: 50] |
| V_{L} | a.a. | NVGTNVA [SEQ ID | SATYRN [SEQ ID NO: | FCQQYNRY [SEQ ID |
| | | NO: 51] | 52] | NO: 53] |

| | |
|---|---|
| Full V_{H} | EVKLQQSGAE LVRPGSSVKI SCKASGYAFS SYWMNWVKQR PGQGLEWIGQ |
| | IYPGDGDTNY NGKFKGQATL TADKSSSTAY MQLSGLTSED SAVYFCARKT |
| | ISSVVDFYFD YWGQGTTVTV SS [SEQ ID NO: 54] |
| Full V_{L} | DIELTQSPKF MSTSVGDRVS VTCKASQNVG TNVAWYQQKP GQSPKPLIYS |
| | ATYRNSGVPD RFTGSGSGTD FTLTITNVQS KDLADYFCQQ YNRYPYTSGG |
| | GTKLEIKR [SEQ ID NO: 55] |
| scFv | MALPVTALLL PLALLLHAEV KLQQSGAELV RPGSSVKISC KASGYAFSSY |
| | WMNWVKQRPG QGLEWIGQIY PGDGDTNYNG KFKGQATLTA DKSSSTAYMQ |
| | LSGLTSEDSA VYFCARKTIS SVVDFYFDYW GQGTTVTVSS GGGGSGGGGS |
| | GGGGSDIELT QSPKFMSTSV GDRVSVTCKA SQNVGTNVAW YQQKPGQSPK |
| | PLIYSATYRN SGVPDRFTGS GSGTDFTLTI TNVQSKDLAD YFCQQYNRYP |
| | YTSGGGTKLE IKR [SEQ ID NO: 60] |
| DNA | ATGGCTCTCCCAGTGACTGCCCTACTGCTTCCCCTAGCGCTTCTCCTGCATGCAGAG |
| | GTGAAGCTGCAGCAGTCTGGGGCTGAGCTGGTGAGGCCTGGGTCCTCAGTGAAGATT |
| | TCCTGCAAGGCTTCTGGCTATGCATTCAGTAGCTACTGGATGAACTGGGTGAAGCAG |
| | AGGCCTGGACAGGGTCTTGAGTGGATTGGACAGATTTATCCTGGAGATGGTGATACT |
| | AACTACAATGGAAAGTTCAAGGGTCAAGCCACACTGACTGCAGACAAATCCTCCAGC |
| | ACAGCCTACATGCAGCTCAGCGGCCTAACATCTGAGGACTCTGCGGTCTATTTCTGT |
| | GCAAGAAAGACCATTAGTTCGGTAGTAGATTTCTACTTTGACTACTGGGGCCAAGGG |
| | ACCACGGTCACCGTCTCCTCAGGTGGAGGTGGATCAGGTGGAGGTGGATCTGGTGGA |
| | GGTGGATCTGACATTGAGCTCACCCAGTCTCCAAAATTCATGTCCACATCAGTAGGA |
| | GACAGGGTCAGCGTCACCTGCAAGGCCAGTCAGAATGTGGGTACTAATGTAGCCTGG |
| | TATCAACAGAAACCAGGACAATCTCCTAAACCACTGATTTACTCGGCAACCTACCGG |
| | AACAGTGGAGTCCCTGATCGCTTCACAGGCAGTGGATCTGGGACAGATTTCACTCTC |
| | ACCATCACTAACGTGCAGTCTAAAGACTTGGCAGACTATTTCTGTCAACAATATAAC |
| | AGGTATCCGTACACGTCCGGAGGGGGGACCAAGCTGGAGATCAAACGG [SEQ ID |
| | NO: 61] |

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to a specific sequence (e.g., SEQ ID NOs: 46, 47, 54, and 55) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 46, 47, 54, and 55). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 46, 47, 54, and 55, including post-translational modifications of that sequence (SEQ ID NO: 46, 47, 54, and 55).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the)(BLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

2.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3t polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide. In certain embodiments, the CD8 polypeptide comprises or has an amino acid sequence that is at least about 85%, about 90%, about 95%, about 96%, about 97%, about 98%, about 99% or about 100% homologous to the sequence having a NCBI Reference No: NP_001139345.1 (SEQ ID NO: 9) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 9 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO: 9. In certain embodiments, the CAR of the presently disclosed comprises a transmembrane domain comprising a CD8 polypeptide that comprises or has an amino acid sequence of amino acids 137 to 209 of SEQ ID NO: 9.

[SEQ ID NO: 9]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the CD8 polypeptide comprises or has an amino acid sequence that is at least about 85%, about 90%, about 95%, about 96%, about 97%, about 98%, about 99% or about 100% homologous to the sequence having a NCBI Reference No: AAA92533.1 (SEQ ID NO: 10) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 10 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 10. In certain embodiments, the CAR of the presently disclosed comprises a transmembrane domain comprising a CD8 polypeptide that comprises or has an amino acid sequence of amino acids 151 to 219 of SEQ ID NO: 10.

| [SEQ ID NO: 10] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the CD8 polypeptide comprises or has the amino acid sequence set forth in SEQ ID NO: 11, which is provided below:

[SEQ ID NO: 11]
STTTKPVLRTPSPVHPTGTSQPQRPEDCRPRGSVKGTGLDFACDIYIWAP
LAGICVALLLSLITTLICY

In accordance with the presently disclosed subject matter, a “CD8 nucleic acid molecule” refers to a polynucleotide encoding a CD8 polypeptide.

In certain embodiments, the CD8 nucleic acid molecule encoding the CD8 polypeptide having the amino acid sequence set forth in SEQ ID NO: 11 comprises or has nucleic acids having the sequence set forth in SEQ ID NO: 12 as provided below.

[SEQ ID NO: 12]
TCTACTACTACCAAGCCAGTGCTGCGAACTCCCTCACCTGTGCACCCTA
CCGGGACATCTCAGCCCCAGAGACCAGAAGATTGTCGGCCCCGTGGCTC
AGTGAAGGGGACCGGATTGGACTTCGCCTGTGATATTTACATCTGGGCA
CCCTTGGCCGGAATCTGCGTGGCCCTTCTGCTGTCCTTGATCATCACTC
TCATCTGCTAC

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID NO: 2), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 2. In certain embodiments, the CD28 polypeptide comprised in the transmembrane domain of a presently disclosed CAR comprises or has an amino acid sequence of amino acids 153 to 179 of SEQ ID NO: 2.

SEQ ID NO: 2 is provided below:

| [SEQ ID NO: 2] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide having amino acids 153 to 179 of SEQ ID NO: 2 comprises or has nucleic acids having the sequence set forth in SEQ ID NO: 22 as provided below.

[SEQ ID NO: 22]
ttttgggtgctggtggtggttggtggagtcctggcttgctatagcttgc
tagtaacagtggcctttattattttctgggtg

In certain embodiments, the intracellular signaling domain of the CAR comprises a murine CD28 transmembrane domain. The murine CD28 transmembrane domain can comprise or have an amino acid sequence that is at least about 85%, about 90%, about 95%, about 96%, about 97%, about 98%, about 99% or about 100% homologous to SEQ ID NO: 73 or fragments thereof, and/or may optionally comprise up to one or up to two or up to three conservative amino acid substitutions. SEQ ID NO: 73 is provided below:

[SEQ ID NO: 73]
FWALVVVAGV LFCYGLLVTV ALCVIWT

An exemplary nucleic acid sequence encoding the amino acid sequence of SEQ ID NO: 73 is set forth in SEQ ID NO: 74, which is provided below.

[SEQ ID NO: 74]
TTTTGGGCACTGGTCGTGGTTGCTGGAGTCCTGTTTTGTTATGGCTTGCT
AGTGACAGTGGCTCTTTGTGTTATCTGGACA

In certain embodiments, the intracellular signaling domain of the CAR comprises a human CD28 transmembrane domain. The human CD28 transmembrane domain can comprise or have an amino acid sequence that is at least about 85%, about 90%, about 95%, about 96%, about 97%, about 98%, about 99% or about 100% homologous to SEQ ID NO: 75 or fragments thereof, and/or may optionally comprise up to one or up to two or up to three conservative amino acid substitutions. SEQ ID NO: 75 is provided below:

[SEQ ID NO: 75]
FWVLVVVGGV LACYSLLVTV AFIIFWV.

An exemplary nucleic acid sequence encoding the amino acid sequence of SEQ ID NO: 75 is set forth in SEQ ID NO: 76, which is provided below.

[SEQ ID NO: 76]
TTTTGGGTGCTGGTGGTGGTTGGTGGAGTCCTGGCTTGCTATAGCTTGCT
AGTAACAGTGGCCTTTATTATTTTCTGGGTG

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 2), a portion of a CD8 polypeptide (e.g., a portion of SEQ ID NO: 9, or a portion of SEQ ID NO: 10), a variation of any of the foregoing which is at least about 80%, at least about 85%, at least about 90%, or at least about 95% homologous thereto, or a synthetic spacer sequence.

2.3.3. Intracellular Signaling Domain of a CAR

In certain non-limiting embodiments, an intracellular signaling domain of the CAR comprises a CD3 polypeptide, which can activate or stimulate a cell (e.g., a cell of the lymphoid lineage, e.g., a T cell). CD3 comprises 3 ITAMs, and transmits an activation signal to the cell (e.g., a cell of the lymphoid lineage, e.g., a T cell) after antigen is bound. The intracellular signaling domain of the CD3-chain is the primary transmitter of signals from endogenous TCRs. In certain embodiments, the CD3 polypeptide comprises or has an amino acid sequence that is at least about 85%, about 90%, about 95%, about 96%, about 97%, about 98%, about 99% or about 100% homologous to the sequence having a NCBI Reference No: NP_932170 (SEQ ID NO: 1), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 1, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 164 of SEQ ID NO: 1. In certain embodiments, the CD3 polypeptide comprises or has an amino acid sequence of amino acids 52 to 164 of SEQ ID NO: 1.

SEQ ID NO: 1 is provided below:

| [SEQ ID NO: 1] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the CD3 polypeptide comprises or has an amino acid sequence that is at least about 85%, about 90%, about 95%, about 96%, about 97%, about 98%, about 99% or about 100% homologous to the sequence having a NCBI Reference No: NP_001106864.2 (SEQ ID NO: 13), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 13, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 13. In certain embodiments, the CD3ζ polypeptide comprises or has an amino acid sequence of amino acids 52 to 142 of SEQ ID NO: 13.

SEQ ID NO: 13 is provided below:

| [SEQ ID NO: 13] | |
|---|---|
| 1 | MKWKVSVLAC ILHVRFPGAE AQSFGLLDPK LCYLLDGILF IYGVIITALY LRAKFSRSAE |
| 61 | TAANLQDPNQ LYNELNLGRR EEYDVLEKKR ARDPEMGGKQ RRRNPQEGVY NALQKDKMAE |
| 121 | AYSEIGTKGE RRRGKGHDGL YQDSHFQAVQ FGNRREREGS ELTRTLGLRA RPKACRHKKP |
| 181 | LSLPAAVS |

In certain embodiments, the CD3 polypeptide comprises or has the amino acid sequence set forth in SEQ ID NO: 14, which is provided below:

[SEQ ID NO: 14]
RAKESRSAETAANLQDPNQLYNELNLGRREEYDVLEKKRARDPEMGGKQ
QRRRNPQEGVYNALQKDKMAEAYSEIGTKGERRRGKGHDGLYQGLSTAT
KDTYDALHMQTLAPR

In accordance with the presently disclosed subject matter, a “CD3 nucleic acid molecule” refers to a polynucleotide encoding a CD3ζ polypeptide. In certain embodiments, the CD3ζ nucleic acid molecule encoding the CD3ζ polypeptide having the amino acid sequence set forth in SEQ ID NO: 14 comprises or has the nucleotide sequence set forth in SEQ ID NO: 15 as provided below.

[SEQ ID NO: 15]
AGAGCAAAATTCAGCAGGAGTGCAGAGACTGCTGCCAACCTGCAGGACC
CCAACCAGCTCTACAATGAGCTCAATCTAGGGCGAAGAGAGGAATATGA
CGTCTTGGAGAAGAAGCGGGCTCGGGATCCAGAGATGGGAGGCAAACAG
CAGAGGAGGAGGAACCCCCAGGAAGGCGTATACAATGCACTGCAGAAAG
ACAAGATGGCAGAAGCCTACAGTGAGATCGGCACAAAAGGCGAGAGGCG
GAGAGGCAAGGGGCACGATGGCCTTTACCAGGGTCTCAGCACTGCCACC
AAGGACACCTATGATGCCCTGCATATGCAGACCCTGGCCCCTCGCTAA

In certain embodiments, the intracellular signaling domain of the CAR comprises a murine CD3ζ polypeptide. The murine CD3ζ polypeptide can comprise or have an amino acid sequence that is at least about 85%, about 90%, about 95%, about 96%, about 97%, about 98%, about 99% or about 100% homologous to SEQ ID NO: 69 or fragments thereof, and/or may optionally comprise up to one or up to two or up to three conservative amino acid substitutions. SEQ ID NO: 69 is provided below:

[SEQ ID NO: 69]
RAKFSRSAET AANLQDPNQL YNELNLGRRE EYDVLEKKRA
RDPEMGGKQQ RRRNPQEGVY NALQKDKMAE AYSEIGTKGE
RRRGKGHDGL YQGLSTATKD TYDALHMQTL APR.

An exemplary nucleic acid sequence encoding the amino acid sequence of SEQ ID NO: 69 is set forth in SEQ ID NO: 70, which is provided below.

[SEQ ID NO: 70]
AGAGCAAAATTCAGCAGGAGTGCAGAGACTGCTGCCAACCTGCAGGACCC
CAACCAGCTCTACAATGAGCTCAATCTAGGGCGAAGAGAGGAATATGACG
TCTTGGAGAAGAAGCGGGCTCGGGATCCAGAGATGGGAGGCAAACAGCAG
AGGAGGAGGAACCCCCAGGAAGGCGTATACAATGCACTGCAGAAAGACAA
GATGGCAGAAGCCTACAGTGAGATCGGCACAAAAGGCGAGAGGCGGAGAG
GCAAGGGGCACGATGGCCTTTACCAGGGTCTCAGCACTGCCACCAAGGAC
ACCTATGATGCCCTGCATATGCAGACCCTGGCCCCTCGC

In certain embodiments, the intracellular signaling domain of the CAR comprises a human CD3ζ polypeptide. The human CD3ζ polypeptide can comprise or have an amino acid sequence that is at least about 85%, about 90%, about 95%, about 96%, about 97%, about 98%, about 99% or about 100% homologous to SEQ ID NO: 71 or fragments thereof, and/or may optionally comprise up to one or up to two or up to three conservative amino acid substitutions. SEQ ID NO: 71 is provided below:

[SEQ ID NO: 71]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR

An exemplary nucleic acid sequence encoding the amino acid sequence of SEQ ID NO: 71 is set forth in SEQ ID NO: 72, which is provided below.

[SEQ ID NO: 72]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain non-limiting embodiments, an intracellular signaling domain of the CAR does not comprise a co-stimulatory signaling region, i.e., the CAR is a first generation CAR.

In certain non-limiting embodiments, an intracellular signaling domain of the CAR further comprises at least a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR′ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide. The CD28 polypeptide can comprise or have an amino acid sequence that is at least about 85%, about 90%, about 95%, about 96%, about 97%, about 98%, about 99% or 100% homologous to the sequence having a NCBI Reference No: P10747 or NP_006130 (SEQ ID NO: 2), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 2. In certain embodiments, the intracellular signaling domain of the CAR comprises a co-stimulatory signaling region that comprises a CD28 polypeptide comprising or having an amino acid sequence of amino acids 180 to 220 of SEQ ID NO: 2.

In certain embodiments, the CD28 polypeptide comprises or has an amino acid sequence that is at least about 85%, about 90%, about 95%, about 96%, about 97%, about 98%, about 99% or about 100% homologous to the sequence having a NCBI Reference No: NP_031668.3 (SEQ ID NO: 16), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 16 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 178 to 218, or 200 to 220 of SEQ ID NO: 16. In certain embodiments, the co-stimulatory signaling region of a presently disclosed CAR comprises a CD28 polypeptide that comprises or has the amino acids 178 to 218 of SEQ ID NO: 16.

SEQ ID NO: 16 is provided below:

| [SEQ ID NO: 16] | |
|---|---|
| 1 | MTLRLLFLAL NFFSVQVTEN KILVKQSPLL VVDSNEVSLSCRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. In certain embodiments, a CD28 nucleic acid molecule that encodes a CD28 polypeptide comprised in the co-stimulatory signaling region of a presently disclosed CAR (e.g., amino acids 178 to 218 of SEQ ID NO: 16) comprises or has a nucleotide sequence set forth in SEQ ID NO: 17, which is provided below.

[SEQ ID NO: 17]
AATAGTAGAAGGAACAGACTCCTTCAAAGTGACTACATGAACATGACTCC
CCGGAGGCCTGGGCTCACTCGAAAGCCTTACCAGCCCTACGCCCCTGCCA
GAGACTTTGCAGCGTACCGCCCC

In certain embodiments, the intracellular signaling domain of the CAR comprises a murine intracellular signaling domain of CD28. The murine intracellular signaling domain of CD28 can comprise or have an amino acid sequence that is at least about 85%, about 90%, about 95%, about 96%, about 97%, about 98%, about 99% or about 100% homologous to SEQ ID NO: 65 or fragments thereof, and/or may optionally comprise up to one or up to two or up to three conservative amino acid substitutions. SEQ ID NO: 65 is provided below:

[SEQ ID NO: 65]
NSRRNRLLQS DYMNMTPRRP GLTRKPYQPY APARDFAAYR P

An exemplary nucleic acid sequence encoding the amino acid sequence of SEQ ID NO: 65 is set forth in SEQ ID NO: 66, which is provided below.

[SEQ ID NO: 66]
AATAGTAGAAGGAACAGACTCCTTCAAAGTGACTACATGAACATGACTCC
CCGGAGGCCTGGGCTCACTCGAAAGCCTTACCAGCCCTACGCCCCTGCCA
GAGACTTTGCAGCGTACCGCCCC

In certain embodiments, the intracellular signaling domain of the CAR comprises a human intracellular signaling domain of CD28. The human intracellular signaling domain of CD28 can comprise or have an amino acid sequence that is at least about 85%, about 90%, about 95%, about 96%, about 97%, about 98%, about 99% or about 100% homologous to SEQ ID NO: 67 or fragments thereof, and/or may optionally comprise up to one or up to two or up to three conservative amino acid substitutions. SEQ ID NO: 67 is provided below:

[SEQ ID NO: 67]
RSKRSRLLHS DYMNMTPRRP GPTRKHYQPY APPRDFAAYR S

An exemplary nucleic acid sequence encoding the amino acid sequence of SEQ ID NO: 67 is set forth in SEQ ID NO: 68, which is provided below.

[SEQ ID NO: 68]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can comprise or have an amino acid sequence that is at least about 85%, about 90%, about 95%, about 96%, about 97%, about 98%, about 99% or about 100% homologous to the sequence having a NCBI Reference No: P41273 or NP_001552 (SEQ ID NO: 3) or fragments thereof, and/or may optionally comprise up to one or up to two or up to three conservative amino acid substitutions.

SEQ ID NO: 3 is provided below:

| [SEQ ID NO: 3] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

In certain embodiments, the intracellular signaling domain of the CAR comprises an intracellular signaling domain of 4-1BB. The intracellular signaling domain of 4-1BB can comprise or have an amino acid sequence that is at least about 85%, about 90%, about 95%, about 96%, about 97%, about 98%, about 99% or about 100% homologous to SEQ ID NO: 63 or fragments thereof, and/or may optionally comprise up to one or up to two or up to three conservative amino acid substitutions. SEQ ID NO: 63 is provided below:

[SEQ ID NO: 63]
KRGRKKLLYI FKQPFMRPVQ TTQEEDGCSC RFPEEEEGGC EL

An exemplary nucleic acid sequence encoding the amino acid sequence of SEQ ID NO: 63 is set forth in SEQ ID NO: 64, which is provided below.

[SEQ ID NO: 64]
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

An OX40 polypeptide can comprise or have an amino acid sequence that is at least about 85%, about 90%, about 95%, about 96%, about 97%, about 98%, about 99% or about 100% homologous to the sequence having a NCBI Reference No: P43489 or NP_003318 (SEQ ID NO: 18), or fragments thereof, and/or may optionally comprise up to one or up to two or up to three conservative amino acid substitutions.

SEQ ID NO: 18 is provided below:

| [SEQ ID NO: 18] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can comprise or have an amino acid sequence that is at least about 85%, about 90%, about 95%, about 96%, about 97%, about 98%, about 99% or about 100% homologous to the sequence having a NCBI Reference No: NP_036224 (SEQ ID NO: 19) or fragments thereof, and/or may optionally comprise up to one or up to two or up to three conservative amino acid substitutions.

SEQ ID NO: 19 is provided below:

| [SEQ ID NO: 19] | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLIGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, a presently disclosed CAR comprises an extracellular antigen-binding domain that binds to CD19 (e.g., murine CD19), a transmembrane domain comprising a CD28 polypeptide, and an intracellular signaling domain comprising a CD3ζ polypeptide (e.g., a murine CD3 polypeptide), wherein the intracellular signaling domain does not comprise a co-stimulatory signaling region, namely, the CAR is a first generation CAR. In certain embodiments, the CAR is designated as “m19mz” (or “am19mz”). In certain embodiments, the CAR (e.g., m19mz) comprises an amino acid sequence that is at least about 85%, about 90%, about 95%, about 96%, about 97%, about 98%, about 99% or about 100% homologous to the amino acid sequence set forth in SEQ ID NO: 5, which is provided below.

[SEQ ID NO: 5]
MASPLTRFLS LNLLLLGESI ILGSGEAEVQ LQQSGAELVR
PGTSVKLSCK VSGDTITFYY MHFVKQRPGQ GLEWIGRIDP
EDESTKYSEK FKNKATLTAD TSSNTAYLKL SSLTSEDTAT
YFCIYGGYYF DYWGQGVMVT VSSGGGGSGG GGSGGGGSDI
QMTQSPASLS TSLGETVTIQ CQASEDIYSG LAWYQQKPGK
SPQLLIYGAS DLQDGVPSRF SGSGSGTQYS LKITSMQTED
EGVYFCQQGL TYPRTFGGGT KLELKRAAAE QKLISEEDLI
EFMYPPPYLD NERSNGTIIH IKEKHLCHTQ SSPKLFWALV
VVAGVLFCYG LLVTVALCVI WTRAKFSRSA ETAANLQDPN
QLYNELNLGR REEYDVLEKK RARDPEMGGK QQRRRNPQEG
VYNALQKDKM AEAYSEIGTK GERRRGKGHD GLYQGLSTAT
KDTYDALHMQ TLAPR

SEQ ID NO: 5 includes a CD8 leader sequence at amino acids 1 to 27, and is able to bind to CD19 (e.g., murine CD19).

An exemplary nucleic acid sequence encoding the amino acid sequence of SEQ ID NO: 5 is set forth in SEQ ID NO: 20, which is provided below.

[SEQ ID NO: 20]
ATGGCCTCACCGTTGACCCGCTTTCTGTCGCTGAACCTGCTGCTGCTGG
GTGAGTCGATTATCCTGGGGAGTGGAGAAGCTGAAGTCCAGCTGCAGCA
GTCTGGGGCTGAGCTTGTGAGACCTGGGACCTCTGTGAAGTTATCTTGC
AAAGTTTCTGGCGATACCATTACATTTTACTACATGCACTTTGTGAAGC
AAAGGCCTGGACAGGGTCTGGAATGGATAGGAAGGATTGATCCTGAGGA
TGAAAGTACTAAATATTCTGAGAAGTTCAAAAACAAGGCGACACTCACT
GCAGATACATCTTCCAACACAGCCTACCTGAAGCTCAGCAGCCTGACCT
CTGAGGACACTGCAACCTATTTTTGTATCTACGGAGGATACTACTTTGA
TTACTGGGGCCAAGGGGTCATGGTCACAGTCTCCTCAGGTGGAGGTGGA
TCAGGTGGAGGTGGATCTGGTGGAGGTGGATCTGACATCCAGATGACAC
AGTCTCCAGCTTCCCTGTCTACATCTCTGGGAGAAACTGTCACCATCCA
ATGTCAAGCAAGTGAGGACATTTACAGTGGTTTAGCGTGGTATCAGCAG
AAGCCAGGGAAATCTCCTCAGCTCCTGATCTATGGTGCAAGTGACTTAC
AAGACGGCGTCCCATCACGATTCAGTGGCAGTGGATCTGGCACACAGTA
TTCTCTCAAGATCACCAGCATGCAAACTGAAGATGAAGGGGTTTATTTC
TGTCAACAGGGTTTAACGTATCCTCGGACGTTCGGTGGCGGCACCAAGC
TGGAATTGAAACGGGCGGCCGCAGAACAGAAACTGATCTCTGAAGAAGA
CCTGATTGAGTTCATGTACCCTCCGCCTTACCTAGACAACGAGAGGAGC
AATGGAACTATTATTCACATAAAAGAGAAACATCTTTGTCATACTCAGT
CATCTCCTAAGCTGTTTTGGGCACTGGTCGTGGTTGCTGGAGTCCTGTT
TTGTTATGGCTTGCTAGTGACAGTGGCTCTTTGTGTTATCTGGACAAGA
GCAAAATTCAGCAGGAGTGCAGAGACTGCTGCCAACCTGCAGGACCCCA
ACCAGCTCTACAATGAGCTCAATCTAGGGCGAAGAGAGGAATATGACGT
CTTGGAGAAGAAGCGGGCTCGGGATCCAGAGATGGGAGGCAAACAGCAG
AGGAGGAGGAACCCCCAGGAAGGCGTATACAATGCACTGCAGAAAGACA
AGATGGCAGAAGCCTACAGTGAGATCGGCACAAAAGGCGAGAGGCGGAG
AGGCAAGGGGCACGATGGCCTTTACCAGGGTCTCAGCACTGCCACCAAG
GACACCTATGATGCCCTGCATATGCAGACCCTGGCCCCTCGCTAA

In certain embodiments, a presently disclosed CAR comprises an extracellular antigen-binding domain that binds to CD19 (e.g., murine CD19), a transmembrane domain comprising a CD28 polypeptide, and an intracellular signaling domain comprising a CD3ζ polypeptide (e.g., a murine CD3ζ polypeptide) and a co-stimulatory signaling region comprising a CD28 polypeptide (e.g., a murine CD28 polypeptide). In certain embodiments, the CAR is designated as “m19m28z” (or “am19m28z”). In certain embodiments, the CAR (e.g., m19m28z) comprises an amino acid sequence that is at least about 85%, about 90%, about 95%, about 96%, about 97%, about 98%, about 99% or about 100% homologous to the amino acid sequence set forth in SEQ ID NO: 6, which is provided below.

[SEQ ID NO: 6]
MASPLTRFLS LNLLLLGESI ILGSGEAEVQ LQQSGAELVR
PGTSVKLSCK VSGDTITFYY MHFVKQRPGQ GLEWIGRIDP
EDESTKYSEK FKNKATLTAD TSSNTAYLKL SSLTSEDTAT
YFCIYGGYYF DYWGQGVMVT VSSGGGGSGG GGSGGGGSDI
QMTQSPASLS TSLGETVTIQ CQASEDIYSG LAWYQQKPGK
SPQLLIYGAS DLQDGVPSRF SGSGSGTQYS LKITSMQTED
EGVYFCQQGL TYPRTFGGGT KLELKRAAAE QKLISEEDLI
EFMYPPPYLD NERSNGTIIH IKEKHLCHTQ SSPKLFWALV
VVAGVLFCYG LLVTVALCVI WTNSRRNRLL QSDYMNMTPR
RPGLTRKPYQ PYAPARDFAA YRPRAKFSRS AETAANLQDP
NQLYNELNLG RREEYDVLEK KRARDPEMGG KQQRRRNPQE
GVYNALQKDK MAEAYSEIGT KGERRRGKGH DGLYQGLSTA
TKDTYDALHM QTLAPR

SEQ ID NO: 6 includes a CD8 leader sequence at amino acids 1 to 27, and is able to bind to CD19 (e.g., murine CD19).

An exemplary nucleic acid sequence encoding the amino acid sequence of SEQ ID NO: 6 is set forth in SEQ ID NO:7, which is provided below.

[SEQ ID NO: 7]
ATGGCCTCACCGTTGACCCGCTTTCTGTCGCTGAACCTGCTGCTGCTGG
GTGAGTCGATTATCCTGGGGAGTGGAGAAGCTGAAGTCCAGCTGCAGCA
GTCTGGGGCTGAGCTTGTGAGACCTGGGACCTCTGTGAAGTTATCTTGC
AAAGTTTCTGGCGATACCATTACATTTTACTACATGCACTTTGTGAAGC
AAAGGCCTGGACAGGGTCTGGAATGGATAGGAAGGATTGATCCTGAGGA
TGAAAGTACTAAATATTCTGAGAAGTTCAAAAACAAGGCGACACTCACT
GCAGATACATCTTCCAACACAGCCTACCTGAAGCTCAGCAGCCTGACCT
CTGAGGACACTGCAACCTATTTTTGTATCTACGGAGGATACTACTTTGA
TTACTGGGGCCAAGGGGTCATGGTCACAGTCTCCTCAGGTGGAGGTGGA
TCAGGTGGAGGTGGATCTGGTGGAGGTGGATCTGACATCCAGATGACAC
AGTCTCCAGCTTCCCTGTCTACATCTCTGGGAGAAACTGTCACCATCCA
ATGTCAAGCAAGTGAGGACATTTACAGTGGTTTAGCGTGGTATCAGCAG
AAGCCAGGGAAATCTCCTCAGCTCCTGATCTATGGTGCAAGTGACTTAC
AAGACGGCGTCCCATCACGATTCAGTGGCAGTGGATCTGGCACACAGTA
TTCTCTCAAGATCACCAGCATGCAAACTGAAGATGAAGGGGTTTATTTC
TGTCAACAGGGTTTAACGTATCCTCGGACGTTCGGTGGCGGCACCAAGC
TGGAATTGAAACGGGCGGCCGCAGAACAGAAACTGATCTCTGAAGAAGA
CCTGATTGAGTTCATGTACCCTCCGCCTTACCTAGACAACGAGAGGAGC
AATGGAACTATTATTCACATAAAAGAGAAACATCTTTGTCATACTCAGT
CATCTCCTAAGCTGTTTTGGGCACTGGTCGTGGTTGCTGGAGTCCTGTT
TTGTTATGGCTTGCTAGTGACAGTGGCTCTTTGTGTTATCTGGACAAAT
AGTAGAAGGAACAGACTCCTTCAAAGTGACTACATGAACATGACTCCCC
GGAGGCCTGGGCTCACTCGAAAGCCTTACCAGCCCTACGCCCCTGCCAG
AGACTTTGCAGCGTACCGCCCCAGAGCAAAATTCAGCAGGAGTGCAGAG
ACTGCTGCCAACCTGCAGGACCCCAACCAGCTCTACAATGAGCTCAATC
TAGGGCGAAGAGAGGAATATGACGTCTTGGAGAAGAAGCGGGCTCGGGA
TCCAGAGATGGGAGGCAAACAGCAGAGGAGGAGGAACCCCCAGGAAGGC
GTATACAATGCACTGCAGAAAGACAAGATGGCAGAAGCCTACAGTGAGA
TCGGCACAAAAGGCGAGAGGCGGAGAGGCAAGGGGCACGATGGCCTTTA
CCAGGGTCTCAGCACTGCCACCAAGGACACCTATGATGCCCTGCATATG
CAGACCCTGGCCCCTCGCTAA

In certain embodiments, a presently disclosed CAR comprises an extracellular antigen-binding domain that binds to CD19 (e.g., human CD19), a transmembrane domain comprising a CD28 polypeptide, and an intracellular signaling domain comprising a CD3ζ polypeptide (e.g., a murine CD3 polypeptide), wherein the intracellular signaling domain does not comprise a co-stimulatory signaling region, namely, the CAR is a first generation CAR. In certain embodiments, the CAR is designated as “ah19mz”. In certain embodiments, the CAR (e.g., ah19mz) comprises an amino acid sequence that is at least about 85%, about 90%, about 95%, about 96%, about 97%, about 98%, about 99% or about 100% homologous to the amino acid sequence set forth in SEQ ID NO: 30, which is provided below.

[SEQ ID NO: 30]
MALPVTALLL PLALLLHAEV KLQQSGAELV RPGSSVKISC
KASGYAFSSY WMNWVKQRPG QGLEWIGQIY PGDGDTNYNG
KFKGQATLTA DKSSSTAYMQ LSGLTSEDSA VYFCARKTIS
SVVDFYFDYW GQGTTVTVSS GGGGSGGGGS GGGGSDIELT
QSPKFMSTSV GDRVSVTCKA SQNVGTNVAW YQQKPGQSPK
PLIYSATYRN SGVPDRFTGS GSGTDFTLTI TNVQSKDLAD
YFCQQYNRYP YTSGGGTKLE IKRAAAIEFM YPPPYLDNER
SNGTIIHIKE KHLCHTQSSP KLFWALVVVA GVLFCYGLLV
TVALCVIWTR AKFSRSAETA ANLQDPNQLY NELNLGRREE
YDVLEKKRAR DPEMGGKQQR RRNPQEGVYN ALQKDKMAEA
YSEIGTKGER RRGKGHDGLY QGLSTATKDT YDALHMQTLA
PR

SEQ ID NO: 30 includes a CD8 leader sequence at amino acids 1 to 18, and is able to bind to CD19 (e.g., human CD19).

An exemplary nucleic acid sequence encoding the amino acid sequence of SEQ ID NO: 30 is set forth in SEQ ID NO: 31, which is provided below.

[SEQ ID NO: 31]
ATGGCTCTCCCAGTGACTGCCCTACTGCTTCCCCTAGCGCTTCTCCTGC
ATGCAGAGGTGAAGCTGCAGCAGTCTGGGGCTGAGCTGGTGAGGCCTGG
GTCCTCAGTGAAGATTTCCTGCAAGGCTTCTGGCTATGCATTCAGTAGC
TACTGGATGAACTGGGTGAAGCAGAGGCCTGGACAGGGTCTTGAGTGGA
TTGGACAGATTTATCCTGGAGATGGTGATACTAACTACAATGGAAAGTT
CAAGGGTCAAGCCACACTGACTGCAGACAAATCCTCCAGCACAGCCTAC
ATGCAGCTCAGCGGCCTAACATCTGAGGACTCTGCGGTCTATTTCTGTG
CAAGAAAGACCATTAGTTCGGTAGTAGATTTCTACTTTGACTACTGGGG
CCAAGGGACCACGGTCACCGTCTCCTCAGGTGGAGGTGGATCAGGTGGA
GGTGGATCTGGTGGAGGTGGATCTGACATTGAGCTCACCCAGTCTCCAA
AATTCATGTCCACATCAGTAGGAGACAGGGTCAGCGTCACCTGCAAGGC
CAGTCAGAATGTGGGTACTAATGTAGCCTGGTATCAACAGAAACCAGGA
CAATCTCCTAAACCACTGATTTACTCGGCAACCTACCGGAACAGTGGAG
TCCCTGATCGCTTCACAGGCAGTGGATCTGGGACAGATTTCACTCTCAC
CATCACTAACGTGCAGTCTAAAGACTTGGCAGACTATTTCTGTCAACAA
TATAACAGGTATCCGTACACGTCCGGAGGGGGGACCAAGCTGGAGATCA
AACGGGCGGCCGCAATTGAGTTCATGTACCCTCCGCCTTACCTAGACAA
CGAGAGGAGCAATGGAACTATTATTCACATAAAAGAGAAACATCTTTGT
CATACTCAGTCATCTCCTAAGCTGTTTTGGGCACTGGTCGTGGTTGCTG
GAGTCCTGTTTTGTTATGGCTTGCTAGTGACAGTGGCTCTTTGTGTTAT
CTGGACAAGAGCAAAATTCAGCAGGAGTGCAGAGACTGCTGCCAACCTG
CAGGACCCCAACCAGCTCTACAATGAGCTCAATCTAGGGCGAAGAGAGG
AATATGACGTCTTGGAGAAGAAGCGGGCTCGGGATCCAGAGATGGGAGG
CAAACAGCAGAGGAGGAGGAACCCCCAGGAAGGCGTATACAATGCACTG
CAGAAAGACAAGATGGCAGAAGCCTACAGTGAGATCGGCACAAAAGGCG
AGAGGCGGAGAGGCAAGGGGCACGATGGCCTTTACCAGGGTCTCAGCAC
TGCCACCAAGGACACCTATGATGCCCTGCATATGCAGACCCTGGCCCCT
CGCTAA

In certain embodiments, a presently disclosed CAR comprises an extracellular antigen-binding domain that binds to CD19 (e.g., human CD19), a transmembrane domain comprising a CD28 polypeptide, and an intracellular signaling domain comprising a CD3ζ polypeptide (e.g., a human CD3ζ polypeptide), wherein the intracellular signaling domain does not comprise a co-stimulatory signaling region, namely, the CAR is a first generation CAR. In certain embodiments, the CAR is designated as “ah19 hz”. In certain embodiments, the CAR (e.g., ah19 hz) comprises an amino acid sequence that is at least about 85%, about 90%, about 95%, about 96%, about 97%, about 98%, about 99% or about 100% homologous to the amino acid sequence set forth in SEQ ID NO: 32, which is provided below.

[SEQ ID NO: 32]
MALPVTALLL PLALLLHAEV KLQQSGAELV RPGSSVKISC
KASGYAFSSY WMNWVKQRPG QGLEWIGQIY PGDGDTNYNG
KFKGQATLTA DKSSSTAYMQ LSGLTSEDSA VYFCARKTIS
SVVDFYFDYW GQGTTVTVSS GGGGSGGGGS GGGGSDIELT
QSPKFMSTSV GDRVSVTCKA SQNVGTNVAW YQQKPGQSPK
PLIYSATYRN SGVPDRFTGS GSGTDFTLTI TNVQSKDLAD
YFCQQYNRYP YTSGGGTKLE IKRAAAIEVM YPPPYLDNEK
SNGTIIHVKG KHLCPSPLFP GPSKPFWVLV VVGGVLACYS
LLVTVAFIIF WVRVKFSRSA DAPAYQQGQN QLYNELNLGR
REEYDVLDKR RGRDPEMGGK PRRKNPQEGL YNELQKDKMA
EAYSEIGMKG ERRRGKGHDG LYQGLSTATK DTYDALHMQA
LPPR

SEQ ID NO: 32 includes a CD8 leader sequence at amino acids 1 to 18, and is able to bind to CD19 (e.g., human CD19).

An exemplary nucleic acid sequence encoding the amino acid sequence of SEQ ID NO: 32 is set forth in SEQ ID NO: 33, which is provided below.

[SEQ ID NO: 33]
ATGGCTCTCCCAGTGACTGCCCTACTGCTTCCCCTAGCGCTTCTCCTGC
ATGCAGAGGTGAAGCTGCAGCAGTCTGGGGCTGAGCTGGTGAGGCCTGG
GTCCTCAGTGAAGATTTCCTGCAAGGCTTCTGGCTATGCATTCAGTAGC
TACTGGATGAACTGGGTGAAGCAGAGGCCTGGACAGGGTCTTGAGTGGA
TTGGACAGATTTATCCTGGAGATGGTGATACTAACTACAATGGAAAGTT
CAAGGGTCAAGCCACACTGACTGCAGACAAATCCTCCAGCACAGCCTAC
ATGCAGCTCAGCGGCCTAACATCTGAGGACTCTGCGGTCTATTTCTGTG
CAAGAAAGACCATTAGTTCGGTAGTAGATTTCTACTTTGACTACTGGGG
CCAAGGGACCACGGTCACCGTCTCCTCAGGTGGAGGTGGATCAGGTGGA
GGTGGATCTGGTGGAGGTGGATCTGACATTGAGCTCACCCAGTCTCCAA
AATTCATGTCCACATCAGTAGGAGACAGGGTCAGCGTCACCTGCAAGGC
CAGTCAGAATGTGGGTACTAATGTAGCCTGGTATCAACAGAAACCAGGA
CAATCTCCTAAACCACTGATTTACTCGGCAACCTACCGGAACAGTGGAG
TCCCTGATCGCTTCACAGGCAGTGGATCTGGGACAGATTTCACTCTCAC
CATCACTAACGTGCAGTCTAAAGACTTGGCAGACTATTTCTGTCAACAA
TATAACAGGTATCCGTACACGTCCGGAGGGGGGACCAAGCTGGAGATCA
AACGGGCGGCCGCAATTGAAGTTATGTATCCTCCTCCTTACCTAGACAA
TGAGAAGAGCAATGGAACCATTATCCATGTGAAAGGGAAACACCTTTGT
CCAAGTCCCCTATTTCCCGGACCTTCTAAGCCCTTTTGGGTGCTGGTGG
TGGTTGGTGGAGTCCTGGCTTGCTATAGCTTGCTAGTAACAGTGGCCTT
TATTATTTTCTGGGTGAGAGTGAAGTTCAGCAGGAGCGCAGACGCCCCC
GCGTACCAGCAGGGCCAGAACCAGCTCTATAACGAGCTCAATCTAGGAC
GAAGAGAGGAGTACGATGTTTTGGACAAGAGACGTGGCCGGGACCCTGA
GATGGGGGGAAAGCCGAGAAGGAAGAACCCTCAGGAAGGCCTGTACAAT
GAACTGCAGAAAGATAAGATGGCGGAGGCCTACAGTGAGATTGGGATGA
AAGGCGAGCGCCGGAGGGGCAAGGGGCACGATGGCCTTTACCAGGGTCT
CAGTACAGCCACCAAGGACACCTACGACGCCCTTCACATGCAGGCCCTG
CCCCCTCGCTAG

In certain embodiments, a presently disclosed CAR comprises an extracellular antigen-binding domain that binds to CD19 (e.g., human CD19), a transmembrane domain comprising a CD28 polypeptide, and an intracellular signaling domain comprising a CD3ζ polypeptide (e.g., a murine CD3ζ polypeptide) and a co-stimulatory signaling region comprising a CD28 polypeptide (e.g., a murine CD28 polypeptide). In certain embodiments, the CAR is designated as “ah19m28z”. In certain embodiments, the CAR (e.g., ah19m28z) comprises an amino acid sequence that is at least about 85%, about 90%, about 95%, about 96%, about 97%, about 98%, about 99% or about 100% homologous to the amino acid sequence set forth in SEQ ID NO: 34, which is provided below.

[SEQ ID NO: 34]
MALPVTALLL PLALLLHAEV KLQQSGAELV RPGSSVKISC
KASGYAFSSY WMNWVKQRPG QGLEWIGQIY PGDGDTNYNG
KFKGQATLTA DKSSSTAYMQ LSGLTSEDSA VYFCARKTIS
SVVDFYFDYW GQGTTVTVSS GGGGSGGGGS GGGGSDIELT
QSPKFMSTSV GDRVSVTCKA SQNVGTNVAW YQQKPGQSPK
PLIYSATYRN SGVPDRFTGS GSGTDFTLTI TNVQSKDLAD
YFCQQYNRYP YTSGGGTKLE IKRAAAIEFM YPPPYLDNER
SNGTIIHIKE KHLCHTQSSP KLFWALVVVA GVLFCYGLLV
TVALCVIWTN SRRNRLLQSD YMNMTPRRPG LTRKPYQPYA
PARDFAAYRP RAKFSRSAET AANLQDPNQL YNELNLGRRE
EYDVLEKKRA RDPEMGGKQQ RRRNPQEGVY NALQKDKMAE
AYSEIGTKGE RRRGKGHDGL YQGLSTATKD TYDALHMQTL
APR

SEQ ID NO: 34 includes a CD8 leader sequence at amino acids 1 to 18, and is able to bind to CD19 (e.g., human CD19).

An exemplary nucleic acid sequence encoding the amino acid sequence of SEQ ID NO: 34 is set forth in SEQ ID NO: 35, which is provided below.

[SEQ ID NO: 35]
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCAAT
TGAGTTCATGTACCCTCCGCCTTACCTAGACAACGAGAGGAGCAATGGAA
CTATTATTCACATAAAAGAGAAACATCTTTGTCATACTCAGTCATCTCCT
AAGCTGTTTTGGGCACTGGTCGTGGTTGCTGGAGTCCTGTTTTGTTATGG
CTTGCTAGTGACAGTGGCTCTTTGTGTTATCTGGACAAATAGTAGAAGGA
ACAGACTCCTTCAAAGTGACTACATGAACATGACTCCCCGGAGGCCTGGG
CTCACTCGAAAGCCTTACCAGCCCTACGCCCCTGCCAGAGACTTTGCAGC
GTACCGCCCCAGAGCAAAATTCAGCAGGAGTGCAGAGACTGCTGCCAACC
TGCAGGACCCCAACCAGCTCTACAATGAGCTCAATCTAGGGCGAAGAGAG
GAATATGACGTCTTGGAGAAGAAGCGGGCTCGGGATCCAGAGATGGGAGG
CAAACAGCAGAGGAGGAGGAACCCCCAGGAAGGCGTATACAATGCACTGC
AGAAAGACAAGATGGCAGAAGCCTACAGTGAGATCGGCACAAAAGGCGAG
AGGCGGAGAGGCAAGGGGCACGATGGCCTTTACCAGGGTCTCAGCACTGC
CACCAAGGACACCTATGATGCCCTGCATATGCAGACCCTGGCCCCTCGCT
GA

In certain embodiments, a presently disclosed CAR comprises an extracellular antigen-binding domain that binds to CD19 (e.g., human CD19), a transmembrane domain comprising a CD28 polypeptide, and an intracellular signaling domain comprising a CD3ζ polypeptide (e.g., a human CD3ζ polypeptide) and a co-stimulatory signaling region comprising a CD28 polypeptide (e.g., a human CD28 polypeptide). In certain embodiments, the CAR is designated as “ah19h28z”. In certain embodiments, the CAR (e.g., ah19h28z) comprises an amino acid sequence that is at least about 85%, about 90%, about 95%, about 96%, about 97%, about 98%, about 99% or about 100% homologous to the amino acid sequence set forth in SEQ ID NO: 36, which is provided below.

[SEQ ID NO: 36]
MALPVTALLL PLALLLHAEV KLQQSGAELV RPGSSVKISC
KASGYAFSSY WMNWVKQRPG QGLEWIGQIY PGDGDTNYNG
KFKGQATLTA DKSSSTAYMQ LSGLTSEDSA VYFCARKTIS
SVVDFYFDYW GQGTTVTVSS GGGGSGGGGS GGGGSDIELT
QSPKFMSTSV GDRVSVTCKA SQNVGTNVAW YQQKPGQSPK
PLIYSATYRN SGVPDRFTGS GSGTDFTLTI TNVQSKDLAD
YFCQQYNRYP YTSGGGTKLE IKRAAAIEVM YPPPYLDNEK
SNGTIIHVKG KHLCPSPLFP GPSKPFWVLV VVGGVLACYS
LLVTVAFIIF WVRSKRSRLL HSDYMNMTPR RPGPTRKHYQ
PYAPPRDFAA YRSRVKFSRS ADAPAYQQGQ NQLYNELNLG
RREEYDVLDK RRGRDPEMGG KPRRKNPQEG LYNELQKDKM
AEAYSEIGMK GERRRGKGHD GLYQGLSTAT KDTYDALHMQ
ALPPR

SEQ ID NO: 36 includes a CD8 leader sequence at amino acids 1 to 18, and is able to bind to CD19 (e.g., human CD19).

An exemplary nucleic acid sequence encoding the amino acid sequence of SEQ ID NO: 36 is set forth in SEQ ID NO: 37, which is provided below.

[SEQ ID NO: 37]
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCAAT
TGAAGTTATGTATCCTCCTCCTTACCTAGACAATGAGAAGAGCAATGGAA
CCATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGGC
TTGCTATAGCTTGCTAGTAACAGTGGCCTTTATTATTTTCTGGGTGAGGA
GTAAGAGGAGCAGGCTCCTGCACAGTGACTACATGAACATGACTCCCCGC
CGCCCCGGGCCCACCCGCAAGCATTACCAGCCCTATGCCCCACCACGCGA
CTTCGCAGCCTATCGCTCCAGAGTGAAGTTCAGCAGGAGCGCAGACGCCC
CCGCGTACCAGCAGGGCCAGAACCAGCTCTATAACGAGCTCAATCTAGGA
CGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATG
AACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAG
TACAGCCACCAAGGACACCTACGACGCCCTTCACATGCAGGCCCTGCCCC
CTCGCTAG

In certain embodiments, a presently disclosed CAR comprises an extracellular antigen-binding domain that binds to CD19 (e.g., human CD19), a transmembrane domain comprising a CD28 polypeptide, and an intracellular signaling domain comprising a CD3ζ polypeptide (e.g., a human CD3ζ polypeptide) and a co-stimulatory signaling region comprising a 4-1BB polypeptide (e.g., a human 4-1BB polypeptide). In certain embodiments, the CAR is designated as “ah19hBBz”. In certain embodiments, the CAR (e.g., ah19hBBz) comprises an amino acid sequence that is at least about 85%, about 90%, about 95%, about 96%, about 97%, about 98%, about 99% or about 100% homologous to the amino acid sequence set forth in SEQ ID NO: 38, which is provided below.

[SEQ ID NO: 38]
MALPVTALLL PLALLLHAEV KLQQSGAELV RPGSSVKISC
KASGYAFSSY WMNWVKQRPG QGLEWIGQIY PGDGDTNYNG
KFKGQATLTA DKSSSTAYMQ LSGLTSEDSA VYFCARKTIS
SVVDFYFDYW GQGTTVTVSS GGGGSGGGGS GGGGSDIELT
QSPKFMSTSV GDRVSVTCKA SQNVGTNVAW YQQKPGQSPK
PLIYSATYRN SGVPDRFTGS GSGTDFTLTI TNVQSKDLAD
YFCQQYNRYP YTSGGGTKLE IKRAAAIEVM YPPPYLDNEK
SNGTIIHVKG KHLCPSPLFP GPSKPFWVLV VVGGVLACYS
LLVTVAFIIF WVKRGRKKLL YIFKQPFMRP VQTTQEEDGC
SCRFPEEEEG GCELRVKFSR SADAPAYQQG QNQLYNELNL
GRREEYDVLD KRRGRDPEMG GKPRRKNPQE GLYNELQKDK
MAEAYSEIGM KGERRRGKGH DGLYQGLSTA TKDTYDALHM
QALPPR

SEQ ID NO: 38 includes a CD8 leader sequence at amino acids 1 to 18, and is able to bind to CD19 (e.g., human CD19).

An exemplary nucleic acid sequence encoding the amino acid sequence of SEQ ID NO: 38 is set forth in SEQ ID NO: 39, which is provided below.

[SEQ ID NO: 39]
ATGGCTCTCCCAGTGACTGCCCTACTGCTTCCCCTAGCGCTTCTCCTGC
ATGCAGAGGTGAAGCTGCAGCAGTCTGGGGCTGAGCTGGTGAGGCCTGG
GTCCTCAGTGAAGATTTCCTGCAAGGCTTCTGGCTATGCATTCAGTAGC
TACTGGATGAACTGGGTGAAGCAGAGGCCTGGACAGGGTCTTGAGTGGA
TTGGACAGATTTATCCTGGAGATGGTGATACTAACTACAATGGAAAGTT
CAAGGGTCAAGCCACACTGACTGCAGACAAATCCTCCAGCACAGCCTAC
ATGCAGCTCAGCGGCCTAACATCTGAGGACTCTGCGGTCTATTTCTGTG
CAAGAAAGACCATTAGTTCGGTAGTAGATTTCTACTTTGACTACTGGGG
CCAAGGGACCACGGTCACCGTCTCCTCAGGTGGAGGTGGATCAGGTGGA
GGTGGATCTGGTGGAGGTGGATCTGACATTGAGCTCACCCAGTCTCCAA
AATTCATGTCCACATCAGTAGGAGACAGGGTCAGCGTCACCTGCAAGGC
CAGTCAGAATGTGGGTACTAATGTAGCCTGGTATCAACAGAAACCAGGA
CAATCTCCTAAACCACTGATTTACTCGGCAACCTACCGGAACAGTGGAG
TCCCTGATCGCTTCACAGGCAGTGGATCTGGGACAGATTTCACTCTCAC
CATCACTAACGTGCAGTCTAAAGACTTGGCAGACTATTTCTGTCAACAA
TATAACAGGTATCCGTACACGTCCGGAGGGGGGACCAAGCTGGAGATCA
AACGGGCGGCCGCAATTGAAGTTATGTATCCTCCTCCTTACCTAGACAA
TGAGAAGAGCAATGGAACCATTATCCATGTGAAAGGGAAACACCTTTGT
CCAAGTCCCCTATTTCCCGGACCTTCTAAGCCCTTTTGGGTGCTGGTGG
TGGTTGGTGGAGTCCTGGCTTGCTATAGCTTGCTAGTAACAGTGGCCTT
TATTATTTTCTGGGTGAAACGGGGCAGAAAGAAACTCCTGTATATATTC
AAACAACCATTTATGAGACCAGTACAAACTACTCAAGAGGAAGATGGCT
GTAGCTGCCGATTTCCAGAAGAAGAAGAAGGAGGATGTGAACTGAGAGT
GAAGTTCAGCAGGAGCGCAGACGCCCCCGCGTACCAGCAGGGCCAGAAC
CAGCTCTATAACGAGCTCAATCTAGGACGAAGAGAGGAGTACGATGTTT
TGGACAAGAGACGTGGCCGGGACCCTGAGATGGGGGGAAAGCCGAGAAG
GAAGAACCCTCAGGAAGGCCTGTACAATGAACTGCAGAAAGATAAGATG
GCGGAGGCCTACAGTGAGATTGGGATGAAAGGCGAGCGCCGGAGGGGCA
AGGGGCACGATGGCCTTTACCAGGGTCTCAGTACAGCCACCAAGGACAC
CTACGACGCCCTTCACATGCAGGCCCTGCCCCCTCGCTAG

The presently disclosed subject matter also provides a nucleic acid composition comprising a first nucleic acid sequence encoding an antigen-recognizing receptor that binds to an antigen and a second nucleic acid sequence encoding an exogenous IL-33 polypeptide.

### 3. Immunoresponsive Cells

The presently disclosed subject matter provides immunoresponsive cells comprising (a) an antigen-recognizing receptor (e.g., CAR or TCR) that binds to an antigen, and (b) a secretable IL-33 polypeptide. In certain embodiments, the secretable IL-33 polypeptide is an exogenous IL-33 polypeptide. In certain embodiments, the antigen-recognizing receptor is capable of activating the immunoresponsive cell. In certain embodiments, the secretable IL-33 polypeptide (e.g., exogenous IL-33 polypeptide, such as a nucleic acid encoding an IL-33 polypeptide) is capable of promoting an anti-tumor effect of the immunoresponsive cell. The immunoresponsive cells can be transduced with an antigen-recognizing receptor and an exogenous IL-33 polypeptide such that the cells co-express the antigen-recognizing receptor and the exogenous IL-33 polypeptide.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

The presently disclosed immunoresponsive cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed immunoresponsive cells have increased secretion of anti-tumor cytokines, including, but not limited to, IL-33, granulocyte macrophage colony-stimulating factor (GM-CSF), IFN-γ, IL-2, IL-5, IL-9, and IL-13. In certain embodiments, the presently disclosed immunoresponsive cells have increased secretion of IL-33, GM-CSF), IFN-γ, IL-2, or a combination thereof

Interleukin-33

Interleukin 33 (IL-33) (also known as DVS27; IL1F11; NF-REV; NFEHEV; C9orf26; GenBank ID: 90865(human), 77125(mouse), 361749(rat), 507054(cattle), 100059908 (horse).) is a gene encoding a cytokine that binds to the IL1RL1/ST2 receptor. IL-33 is involved in the maturation of certain type of T cells and the activation of other immunoresponsive cells, such as mast cells, basophils, eosinophils and natural killer cells. The protein product of IL-33 includes, but is not limited to, NCBI Reference Sequences NP_001186569.1, NP_001186570.1, NP_001300973.1, NP_001300974.1, NP_001300975.1, NP_001300976.1, NP_001300977.1, NP_001340731.1, NP_254274.1, XP 016870774.1 and XP 011516363.1.

In certain embodiments, the term “IL-33” or “IL-33 cytokine” refers to the bioactive form of IL-33 after secretion from a cell (e.g., a form where the signal peptide is cleaved off). A non-limiting example of human IL-33 has the following amino acid sequence set forth in SEQ ID NO: 4, which is provided below.

(SEQ ID NO: 4)
SITGISPITEYLASLSTYNDQSITFALEDESYETYVEDLKKDEKKDKVL
LSYYESQHPSNESGDGVDGKMLMVTLSPTKDFWLHANNKEHSVELHKCE
KPLPDQAFFVLHNMHSNCVSFECKTDPGVFIGVKDNHLALIKVDSSENL
CTENILFKLSET

In certain embodiments, a murine IL-33 polypeptide comprises or has the amino acid sequence set forth in SEQ ID NO: 21, which is provided below. In certain embodiments, a murine IL-33 polypeptide comprises or has an amino acid sequence that is at least about 80%, at least about 85%, at least about 90%, at least about 95%, at least about 99%, or at least about 100% homologous or identical to the sequence set forth in SEQ ID NO: 21.

[SEQ ID NO: 21]
SIQGTSLLTQSPASLSTYNDQSVSFVLENGCYVINVDDSGKOQEQDQVL
LRYYESPCPASQSGDGVDGKKLMVNMSPIKDTDIWLHANDKDYSVELQR
GDVSPPEQAFFVLHKKSSDEVSFECKNLPGTYIGVKDNQLALVEEKDES
CNNIMFKLSKI

In certain embodiments, a secretable IL-33 polypeptide refers to a polypeptide or a protein, the cytokine portion of which has at least about 80%, about 85%, about 90%, about 95%, about 96%, about 97%, about 98%, about 99%, or about 100% homologous to the cytokine portion of the protein product of IL-33 (GenBank ID: 26525 (human), 54450 (mouse), 311783 (rat), 518514 (cattle), 611869 (dog), 100065154 (horse)), or a fragment thereof that has immunostimulatory activity. In certain non-limiting embodiments, the secretable IL-33 polypeptide comprises a cytokine portion and a signal peptide, optionally joined by a linker peptide. Non-limiting examples of secretable IL-33 polypeptides include NCBI Reference Sequences NP_001186569.1, NP_001186570.1, NP_001300973.1, NP_001300974.1, NP_001300975.1, NP_001300976.1, NP_001300977.1, NP_001340731.1, NP_254274.1, XP 016870774.1 and XP 011516363.1.

In certain non-limiting embodiments, the secretable IL-33 polypeptide comprises a signal peptide, for example, an IL-2 signal peptide, a kappa leader sequence, a CD8 leader sequence or a peptide with essentially equivalent activity. In certain embodiments, the secretable IL-33 polypeptide comprises an IL-2 signal peptide. In certain embodiments, the IL-2 signal peptide comprises or has the amino acid sequence set forth in SEQ ID NO: 8

In certain embodiments, the presently disclosed immunoresponsive cells are capable of inducing prolonged B-cell aplasia. In certain embodiments, the immunoresponsive cells comprising an antigen-recognizing receptor and a secretable IL-33 polypeptide (e.g., an exogenous IL-33 polypeptide) decrease B-cell population by at least about 5%, about 10%, about 20%, about 30%, about 40%, about 50%, about 60%, about 70%, about 80%, about 90%, or about 100%, compared to immunoresponsive cells comprising an antigen-recognizing receptor alone (e.g., not comprising a secretable IL-33 polypeptide).

In certain embodiments, the presently disclosed immunoresponsive cells are capable of activating endogenous immune cells. In certain embodiments, the endogenous immune cells are selected from the group consisting of NK cells, NK-T cells, dendritic cells and endogenous CD8 T cells. In certain embodiments, the immunoresponsive cells disclosed herein increase the endogenous immune cells population. In certain embodiments, the immunoresponsive cells comprising an antigen-recognizing receptor and a secretable IL-33 polypeptide (e.g., an exogenous IL-33 polypeptide) increase the endogenous immune cells population by at least about 5%, about 10%, about 20%, about 30%, about 40%, about 50%, about 60%, about 70%, about 80%, about 90%, about 100%, about 150%, about 200%, about 250%, about 300%, about 400%, about 500%, about 600%, about 700%, about 800%, about 900%, about 1000%, or more compared to immunoresponsive cells comprising an antigen-recognizing receptor alone (e.g., not comprising a secretable IL-33 polypeptide).

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells, e.g., at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

### 4. Vectors

Genetic modification of an immunoresponsive cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include antigen recognizing receptors (e.g., CARs or TCRs), a retroviral vector is generally employed for transduction, however any other suitable viral vector or non-viral delivery system can be used. The antigen-recognizing receptor and the IL-33 polypeptide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease Fokl. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### 5. Enhancing Endogenous IL-33 Gene Expression

Any targeted genome editing methods can be used to modify the promoter/enhancer region of an IL-33 gene locus, and thereby enhancing the endogenous expression of IL-33 in an immunoresponsive cell. In certain embodiments, the modification comprises replacement of an endogenous promoter with a constitutive promoter or an inducible promoter, or insertion of a constitutive promoter or inducible promoter to the promoter region of an IL-33 gene locus. In certain embodiments, a constitutive promoter is positioned on an IL-33 gene locus to drive gene expression of the endogenous IL-33 gene. Eligible constitutive promoters include, but are not limited to, a CMV promoter, an EF1a promoter, a SV40 promoter, a PGK1 promoter, a Ubc promoter, a beta-actin promoter, and a CAG promoter. Alternatively or additionally, a conditional or inducable promoter is positioned on an IL-33 gene locus to drive gene expression of the endogenous IL-33 gene. Non-limiting examples of conditional promoters include a tetracycline response element (TRE) promoter and an estrogen response element (ERE) promoter. In addition, enhancer elements can be placed in regions other than the promoter region.

### 6. Genome Editing Methods

Any targeted genome editing methods can be used to modify the promoter/enhancer region of an IL-33 gene locus. In certain embodiments, a CRISPR system is used to modify the promoter/enhancer region of an IL-33 gene locus. In certain embodiments, zinc-finger nucleases are used to modify the promoter/enhancer region of an IL-33 gene locus. In certain embodiments, a TALEN system is used to modify the promoter/enhancer region of an IL-33 gene locus.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within an IL-33 gene locus, or anywhere that can impact gene expression of an IL-33 gene. In certain embodiments, the modification occurs upstream of the transcriptional start site of an IL-33 gene. In certain embodiments, the modification occurs between the transcriptional start site and the protein coding region of an IL-33 gene. In certain embodiments, the modification occurs downstream of the protein coding region of an IL-33 gene. In certain embodiments, the modification occurs upstream of the transcriptional start site of an IL-33 gene, wherein the modification produces a new transcriptional start site.

### 7. Polypeptides and Analogs

Also included in the presently disclosed subject matter are a CD19, CD28, 4-1BB. CD3ζ, and IL-33 polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD28, CD3ζ, and IL-33). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and C⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., IL-33). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 8. Administration

Compositions comprising the presently disclosed immunoresponsive cells can be provided systemically or directly to a subject for inducing and/or enhancing an immune response to an antigen and/or treating and/or preventing a neoplasm, pathogen infection, or infectious disease. In certain embodiments, the presently disclosed immunoresponsive cells or compositions comprising thereof are directly injected into an organ of interest (e.g., an organ affected by a neoplasm). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. certain embodiments, the purity is about 70% to about 75%, about 75% to about 80%, or about 80% to about 85%. certain embodiments, the purity is about 85% to about 90%, about 90% to about 95%, or about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 9. Formulations

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride is particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁵ and about 10⁹, or between about 10⁶ and about 10⁸ of the presently disclosed immunoresponsive cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed immunoresponsive cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 10. Methods of Treatment

The presently disclosed subject matter provides methods for inducing and/or increasing an immune response in a subject in need thereof. The presently disclosed immunoresponsive cells and compositions comprising thereof can be used for treating and/or preventing a neoplasm in a subject. The presently disclosed immunoresponsive cells and compositions comprising thereof can be used for prolonging the survival of a subject suffering from a neoplasm. The presently disclosed immunoresponsive cells and compositions comprising thereof can also be used for treating and/or preventing a pathogen infection or other infectious disease in a subject, such as an immunocompromised human subject. Such methods comprise administering the presently disclosed immunoresponsive cells in an amount effective or a composition (e.g., pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁶-10¹⁰ (e.g., about 10) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

The presently disclosed subject matter provides methods for treating and/or preventing a neoplasm in a subject. The method can comprise administering an effective amount of the presently disclosed immunoresponsive cells or a composition comprising thereof to a subject having a neoplasm.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

As a consequence of surface expression of an antigen-recognizing receptor that binds to a tumor antigen and a secretable IL-33 polypeptide (e.g., an exogenous IL-33 polypeptide) that enhances the anti-tumor effect of the immunoresponsive cell, adoptively transferred T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

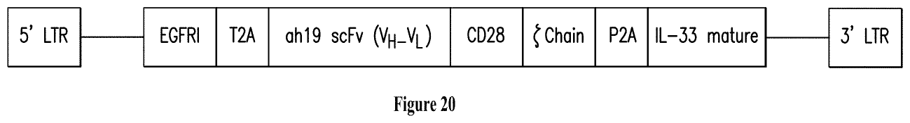

Further modification can be introduced to the presently disclosed immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed immunoresponsive cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. An exemplary embodiment is shown in FIG. 20. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CART cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

### 11. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed immunoresponsive cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding an IL-33 polypeptide in expressible (and secretable) form, which may optionally be comprised in the same or different vectors.

If desired, the immunoresponsive cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of a neoplasm or a pathogen infection. In certain embodiments, the instructions include at least one of the following: description of the therapeutic agent; dosage schedule and administration for treatment or prevention of a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1— Interleukin-33 (IL-33) Secreting CAR-T Cells

Introduction

Genetically modified T cells expressing a first-generation anti-CD19 CAR and a secretable IL-33 polypeptide or a second-generation anti-CD19 CAR and a secretable a murine IL-33 polypeptide were generated. The IL-33-secreting CAR-T cells presented improvements when compared to a control anti-CD19 CAR-T cells that do not comprise a secretable IL-33 polypeptide. The IL-33-secreting CAR-T cells exhibited prolonged survival curves in murine models.

Results

CAR Constructs

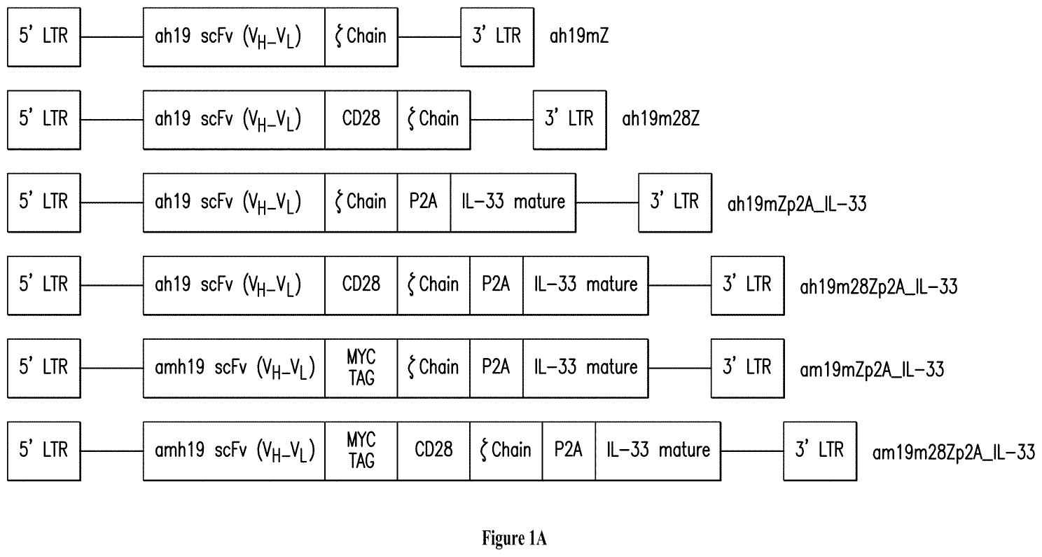

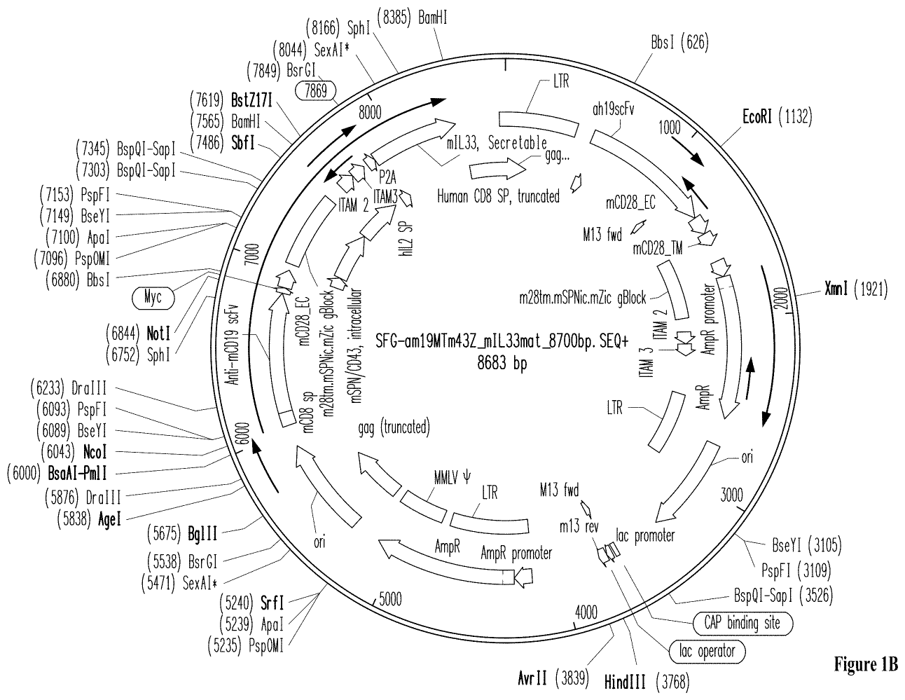

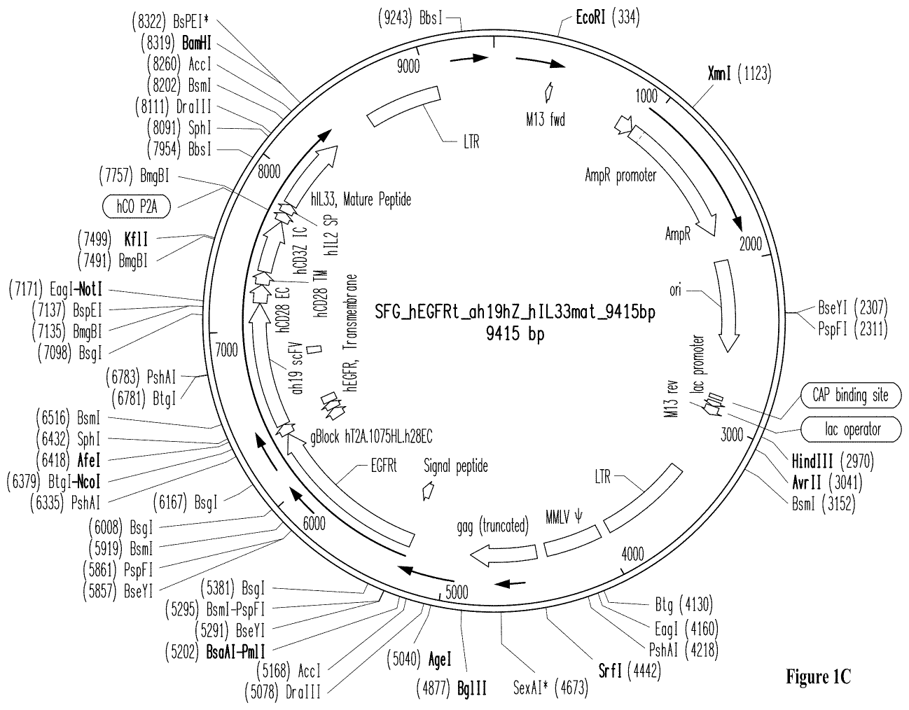

Various anti-CD19 CAR constructs with or without a secretable murine IL-33 polypeptide were constructed in SFG retroviral vector backbone, e.g., ah19mZ, ah19m28Z, ah19mZp2A_IL-33, ah19m28Z, ah19m28Zp2A_IL-33, am19m28Z, and am19m28Zp2A_IL-33, as shown in FIG. 1A.

FIG. 1B shows the construct of first-generation anti-mouse CD19 myc-tag CAR incorporating constitutively-secreted murine IL-33, wherein the amino acid sequence of murine IL-33 mature peptide used in the construct is set forth in SEQ ID NO: 21.

FIG. 1C shows the construct of first-generation anti-human CD 19 (SJ25C scFv) CAR incorporating constitutively-secreted human IL-33, wherein the amino acid sequence of human IL-33 mature peptide used in the construct is set forth in SEQ ID NO: 4.

T cells were transduced with one of the above-noted various constructs.

Increased Cytokine Secretion

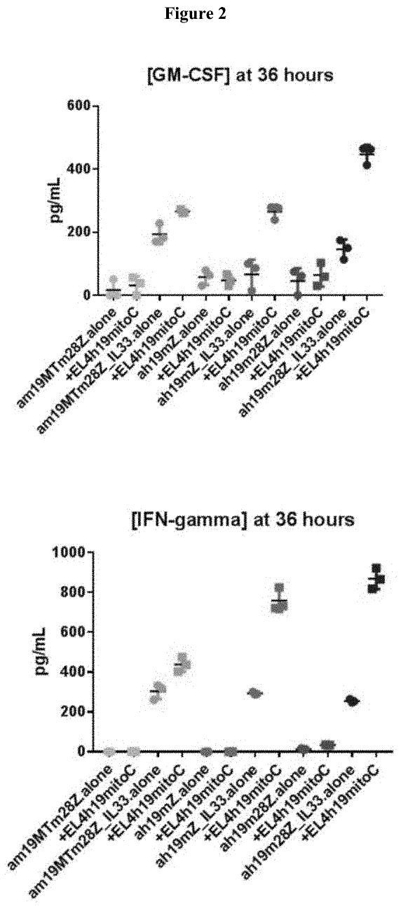

Cytokine secretion of the modified T cells was analyzed by flow cytomatry and the results are shown in FIG. 2. CAR-T cells were cocultured alone or with antigen-positive tumor cells, +EL4h 19mitoC (EL4h 19 cells exposed to mitomycin C to prevent proliferation during assay), at an effector:tumor ratio of 10:1. 36 hours later, supernatant was collected and granulocyte macrophage colony-stimulating factor (GM-CSF) and interferon gamma (IFN-gamma) were measured utilizing a bead-based multiplex assay. IL-33-secreting CAR-T cells showed increased secretion of GM-CSF and IFN-gamma when cocultured alone and with antigen-positive tumor cells as compared to T cells expressing a CAR only without a secretable IL-33 polypeptide (see FIG. 2).

Increased In Vitro Cytotoxicity

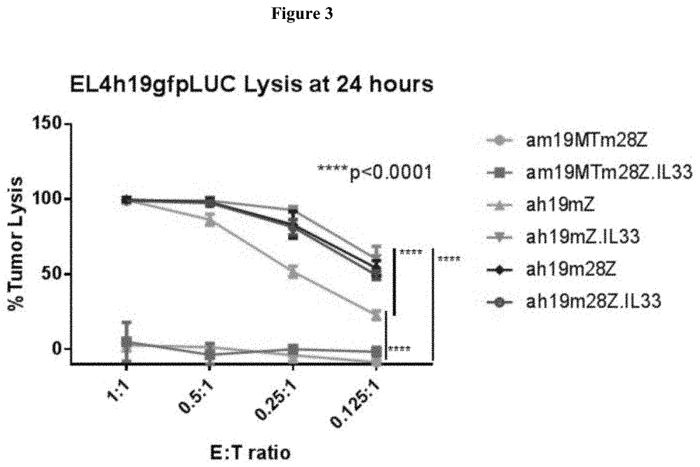

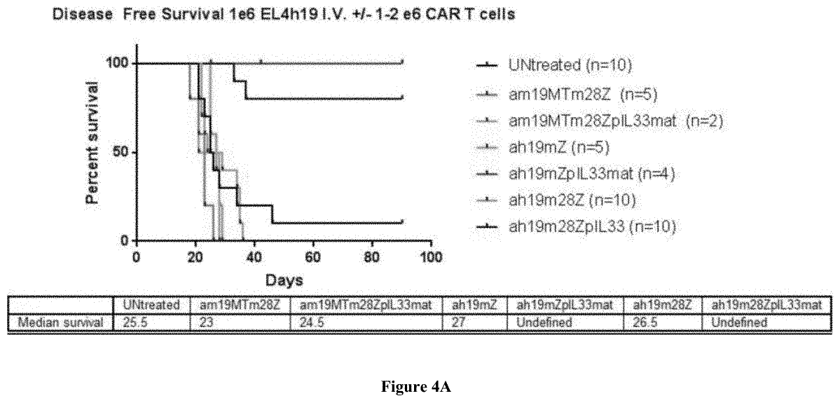

In vitro cytotoxicity of the modified T cells was analyzed. CAR T-cells were cocultured with antigen-positive (human CD19⁺) tumor cells (EL4h19gfpLUC) at different effector:tumor ratios (E:T ratios). Tumor cell lysis was measured by bioluminescence 24 hours later and the results are shown in FIG. 3. As shown in FIG. 3, shows that the in vitro cytotoxicity of IL-33-secreting first generation CAR T cells (e.g., ah19mZ.IL33) was higher than that of the first generation CAR T cells without a secretable IL-33 polypeptide (e.g., ah19mZ), and the in vitro cytotoxicity of IL-33 secreting second generation CAR T cells (e.g., ah19m28Z.IL33) are similar to that of the second generation CAR T cells without a secretable IL-33 polypeptide (e.g., ah19m28Z).

Survival of Tumor-Bearing Mice

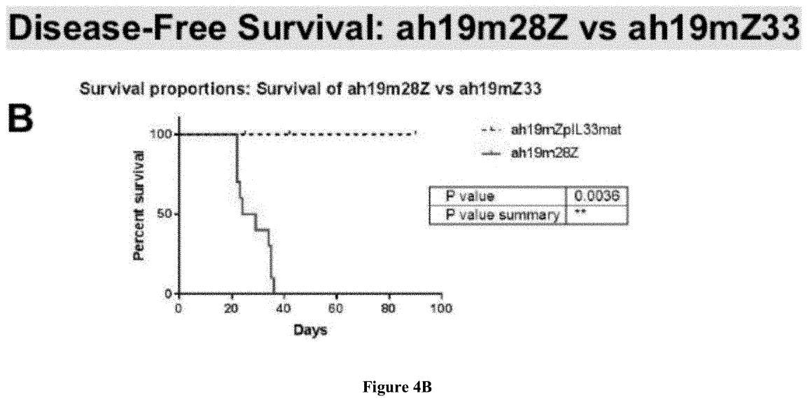

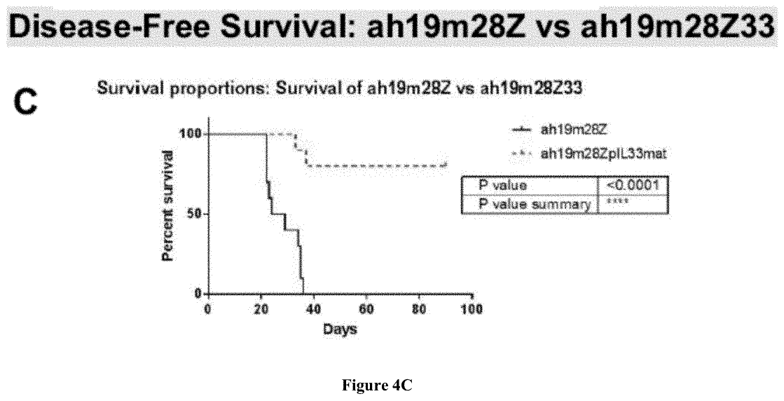

The modified T cells were next studied in the context of syngeneic, immune competent models of disease wherein long-term survival of EL4hCD19⁺ tumor-bearing syngeneic immuno-competent mice were assessed. C57BL/8 mice were inoculated with 1×10⁶ EL4 tumor cells from Sigma which ectopically expressed human CD19 (EL4h19). The mice were subsequently treated with 1×10⁶ to 2×10⁶ CAR T cells transduced with various CAR constructs one day after tumor inoculation, and survival was followed. Survival curves of all subjects are shown in FIGS. 4A-4C. As shown in FIGS. 4A-4C, both IL-33-secreting first generation CAR T cells and IL-33-secreting second generation CAR T cells (ah19mZpIL33mat and ah19m28ZpIL33) induced long-term survival in tumor-bearing mice.

B-Cell Aplasia

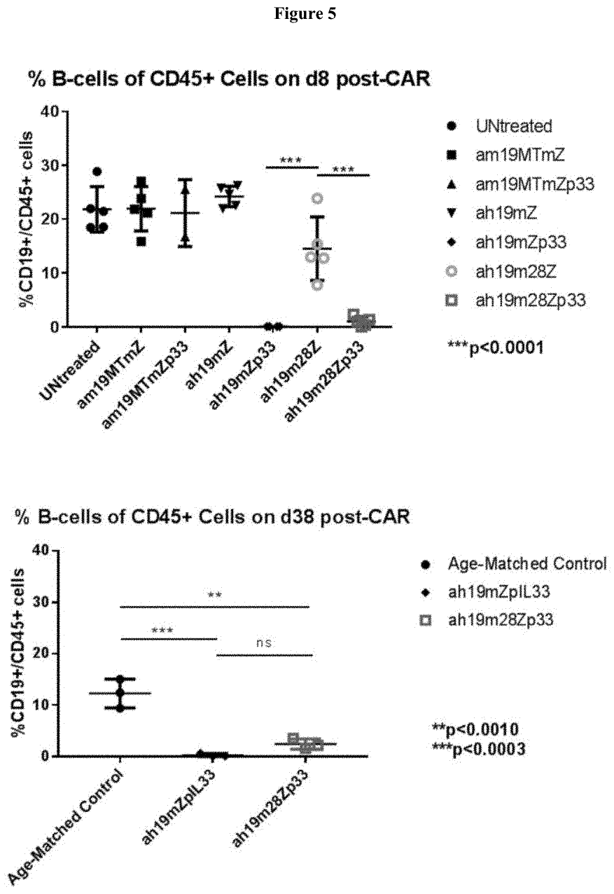

The effects of the modified T cells to B-cell population were analyzed. EL4h19-bearing C57BU6 mice were treated with 2×10⁶ CAR T cells 1 day post tumor inoculation. At day 8, peripheral blood was assessed for B-cells by flow cytometry, and quantified as a percentage of CD45⁺ cells. Measurements were repeated on day 38 in surviving mice, with age-matched controls. As shown in FIG. 5, the IL-33-secreting CAR T cell treatment led to a depletion of B-cells on both day 8 and day 38 in surviving mice. Thus, IL-33-secreting CAR T cells induced a rapid and protracted B-cell aplasia.

Peripheral Distribution of CD11b⁺ Cells

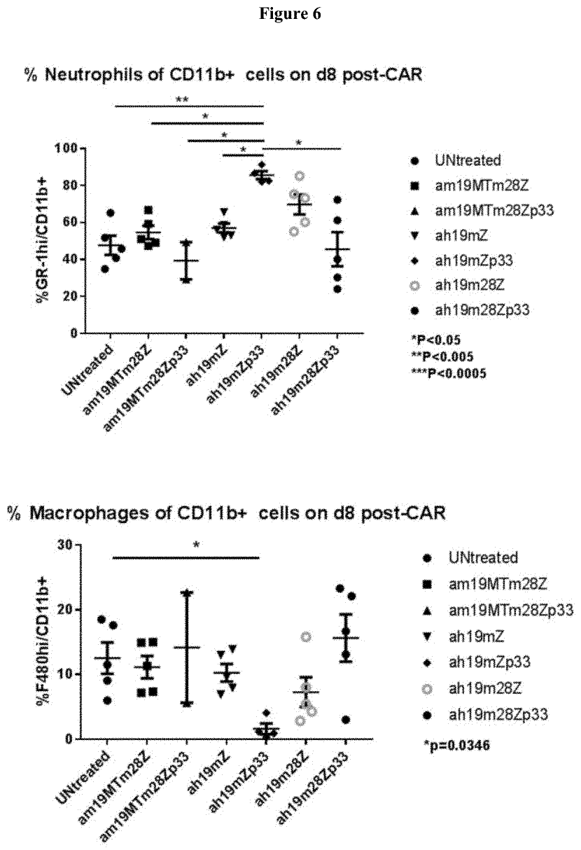

The effects of the modified T cells to CD11b⁺ neutrophils and macrophages were analyzed. EL4h 19-bearing C57BU6 mice were treated with 2×10⁶ CAR T cells 1 day post tumor inoculation. At day 8, peripheral blood was assessed for neutrophils (Gr-lhi) and macrophages (F4/80hi) by flow cytometry, and quantified as a percentage of CD11b⁺ cells. As shown in FIG. 6, the IL-33-secreting first generation CAR T cells treatment increased the population of CD11b⁺ neutrophils and decreased the population of CD11b⁺ macrophages compared to untreated control group.

Peripheral Cytokine Levels

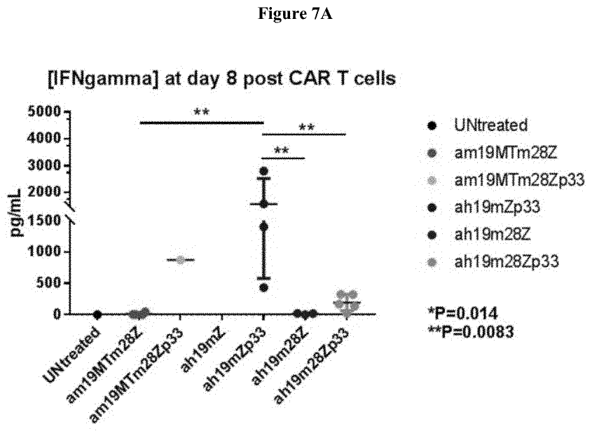

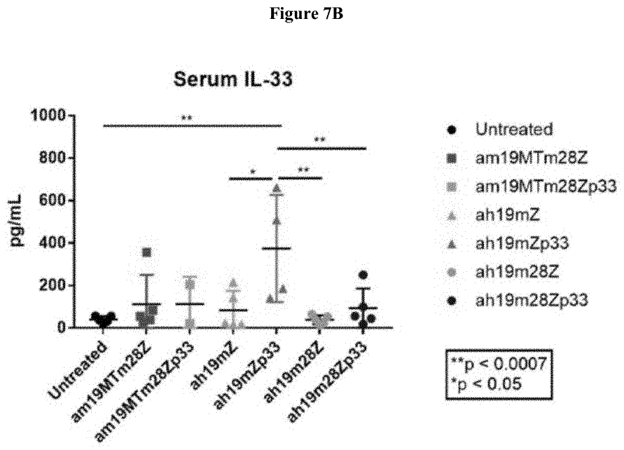

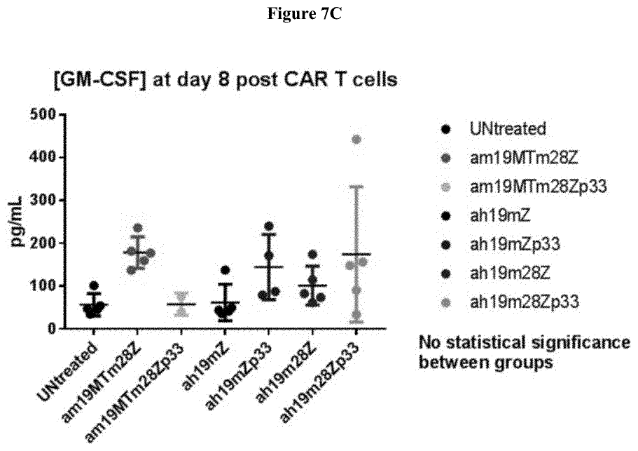

The effects of the modified T cells to cytokine levels in blood were analyzed. EL4h19-bearing C57BL/6 mice were treated with 2×10⁶ CAR T cells 1 day post tumor inoculation. At day 8, peripheral blood was collected and cytokines quantified utilizing a bead-based multiplex assay. As shown in FIG. 7, the IL-33-secreting CAR T cells treatment led to increases in serum levels of interferon gamma, GM-CSF and IL-33 compared to control groups.

### Example 2— Interleukin-33 (IL-33) Secreting CAR-T Cells in Mouse Model

Murine IL-33 Secreting CAR Constructs

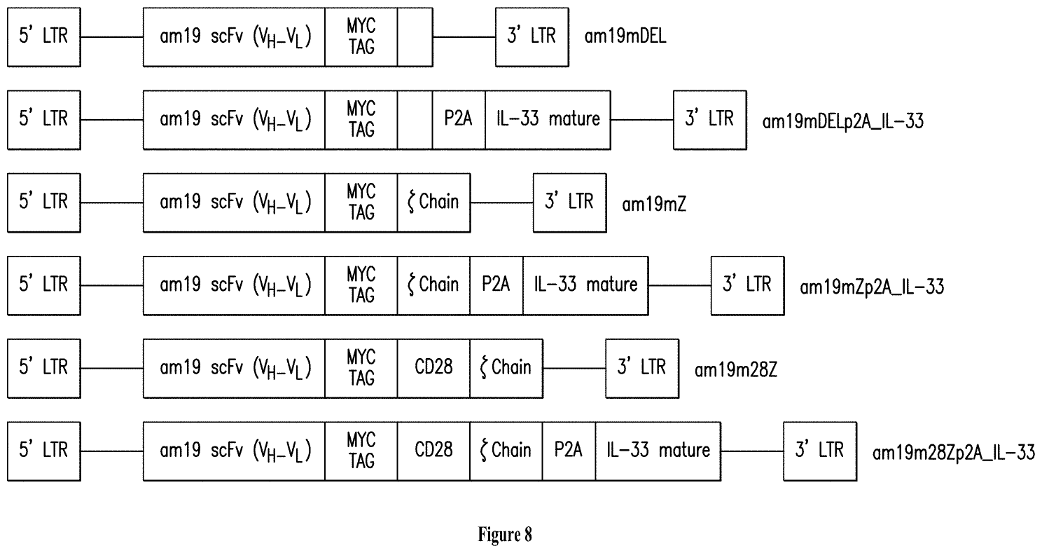

The biological effects of IL-33-secreting CAR-T cells were analyzed in a mouse model. Various anti-murine CD19 CAR constructs with or without a secretable murine IL-33 polypeptide were constructed in SFG retroviral vector backbone as shown in FIG. 8, including am19mDEL, am19mDELp2A_IL-33, am19mZ, am19mZp2A_IL-33, am19m28Z, and am19m28Zp2A_IL-33. Anti-murine CD19 (am19) was derived from 1D3 scFv. All constructs utilized a CD28 proximal extracellular and transmembrane domain as a hinge. In all murine IL-33 secreting constructs, the cytokine was separated from the CAR by a self-cleaving P2A element.

Increased Cytokine Secretion

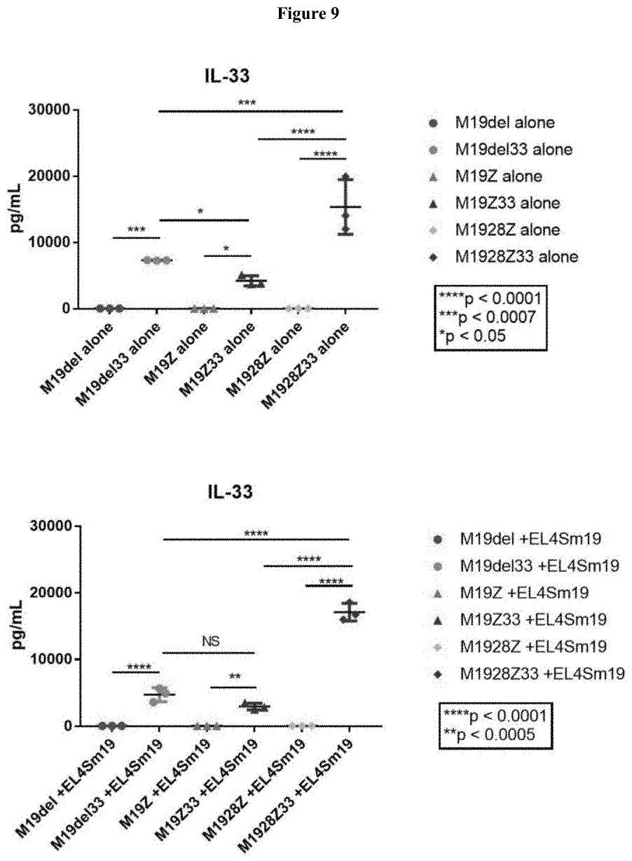

IL-33 secretion was analyzed among T cells transduced with one of the above-noted constructs. These modified T cells were co-cultured alone or with antigen-positive tumor cells, +EL4h19Sm19 (EL4 cells purchased from Sigma, with murine knocked in), at an effector:tumor ratio of 1:1. 24 hours later, supernatant was collected and cytokines were measured utilizing a bead-based multiplex assay. As shown in FIG. 9, detectable levels of soluble IL-33 were observed in IL-33-Secreting CAR T Cells.

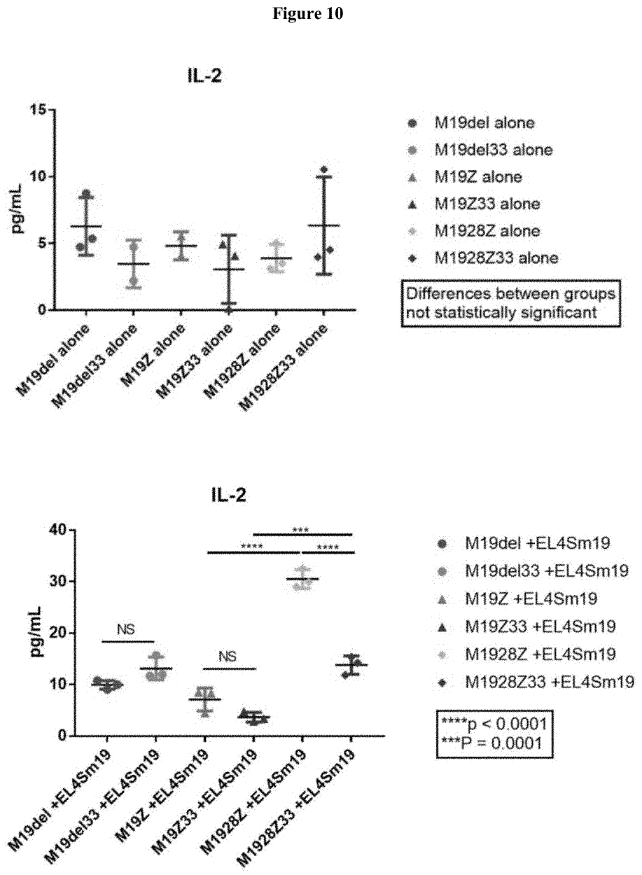

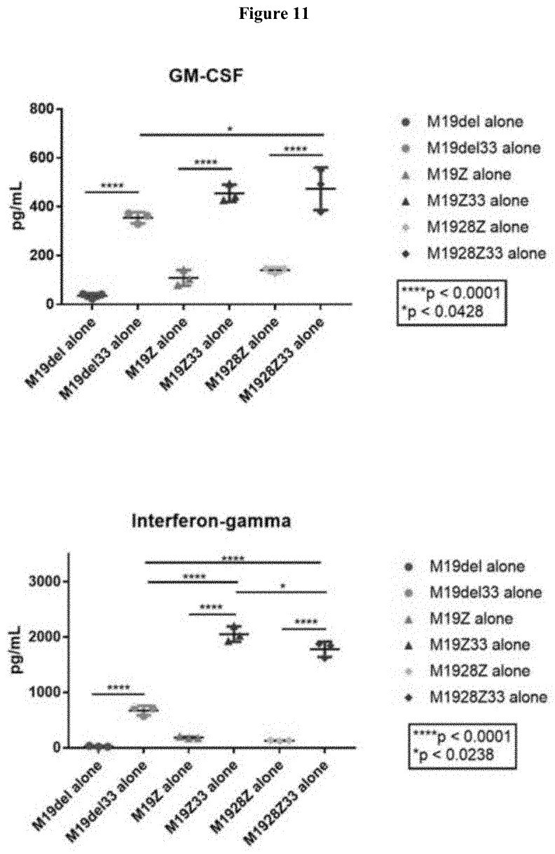

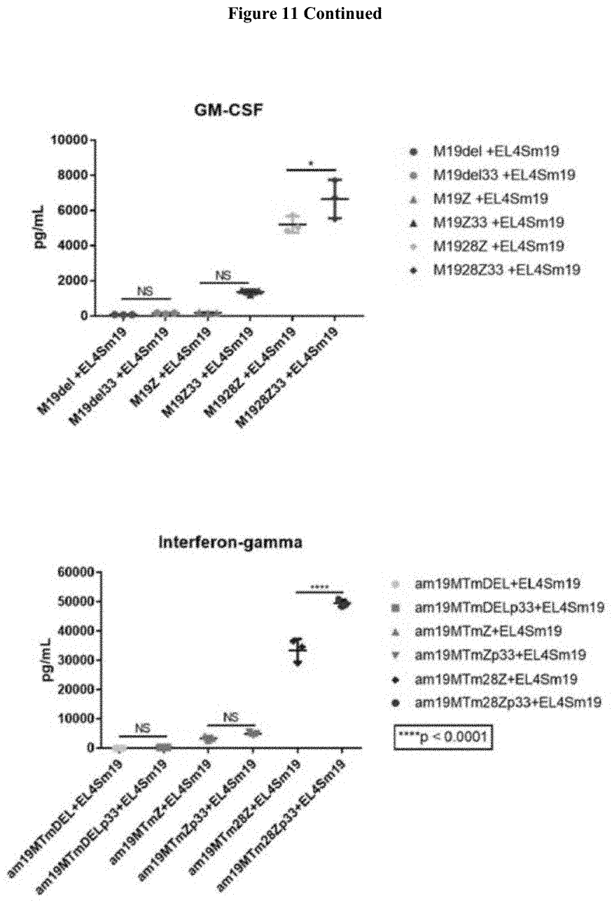

Secretion of other anti-tumor cytokines was also assessed. The modified T cells were cocultured alone or with antigen-positive tumor cells, +EL4h19Sm19 (EL4 cells purchased from Sigma, with murine knocked in), at an effector:tumor ratio of 1:1. 24 hours later, supernatant was collected and cytokines were measured utilizing a bead-based multiplex assay. As shown in FIG. 10, the second-generation IL-33-secreting CAR T cells secreted IL-2 upon stimulation, demonstrating functionality of chimeric costimulatory domain. As shown in FIG. 11, the shows the IL-33-secreting CAR T cells secreted GM-CSF and interferon-gamma independent of antigen stimulation, and this secretion was further enhanced in the presence of antigen and a costimulatory domain.

B-Cell Aplasia

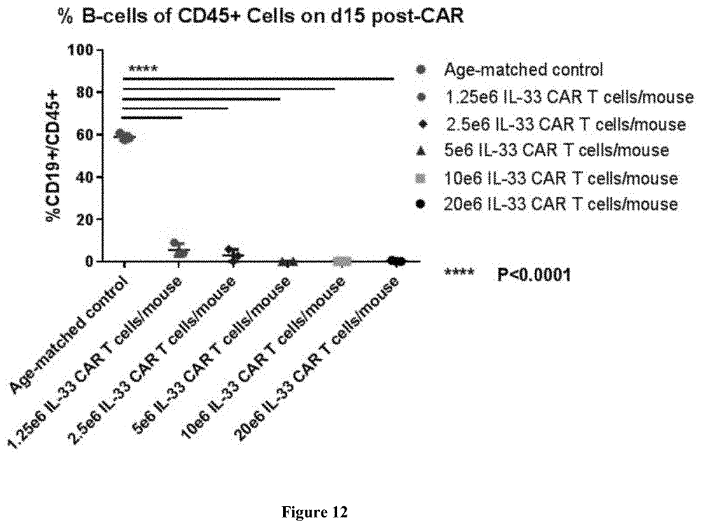

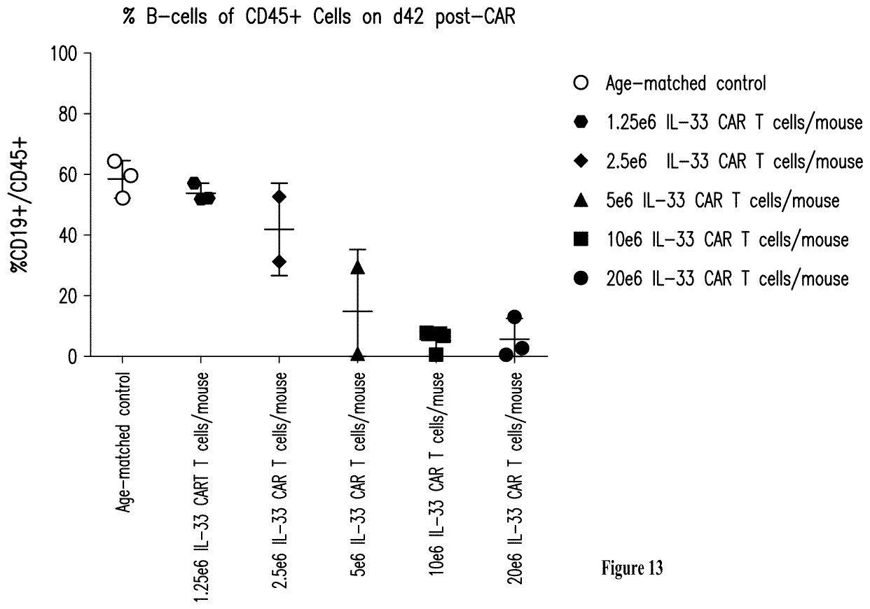

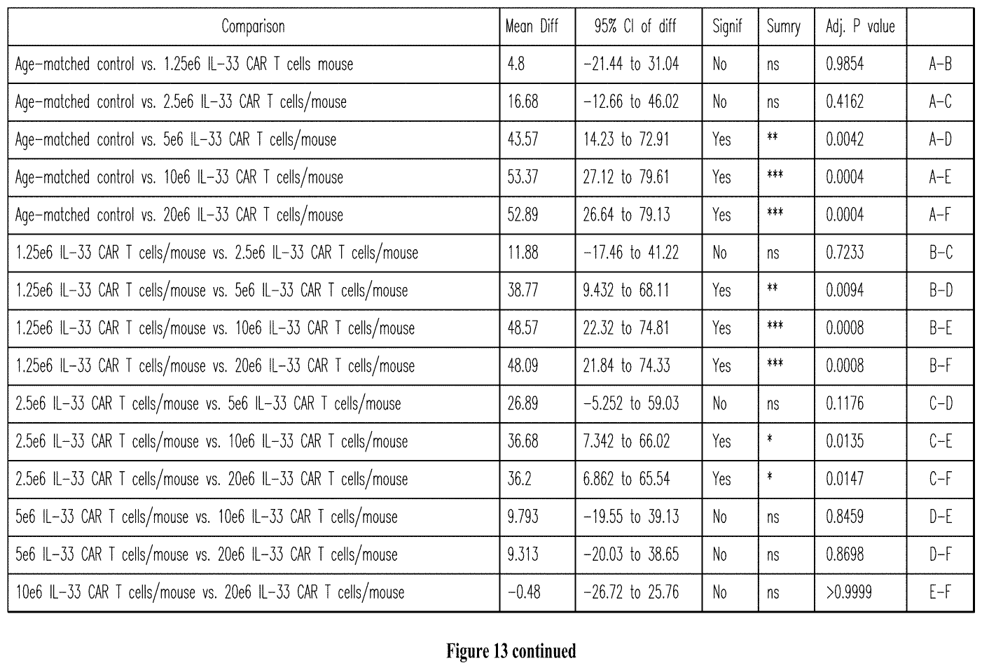

The effects of the modified T cells (am19mZ-IL33) to B-cell population were analyzed. Escalating doses (1.25×10⁶ to 20×10⁶ per mouse) of modified T cells were administered to healthy non-tumor-bearing C57BL/6 mice, and on day 15, peripheral blood was assessed for B-cells by flow cytometry, and quantified as a percentage of CD45⁺ cells. In addition, B-cell recovery was followed. On day 42, peripheral blood was assessed for B-cells by flow cytometry, and quantified as a percentage of CD45+ cells. As shown in FIG. 12, CD45⁺ B-cell population was significantly decreased after the IL-33-secreting CAR T cells (am19mZ-IL33) treatment. The results support that the IL-33-secreting CAR T cells induced B-cell aplasia in the absence of conditioning chemotherapy. As shown in FIG. 13, B-cell recovery post treatment with IL-33-secreting first-generation CAR (am19mZ-IL33) T cells is dose-dependent. B-cell population was significantly recovered in mice treated with lower dose of IL-33 secreting CAR T cells (e.g., 1.25×10⁶ and 2.5×10⁶ T cells) compared to mice treated with higher dose of IL-33 secreting CAR T cells (e.g., 10×10⁶ and 20×10⁶ T cells).

Survival of Tumor-Bearing Mice

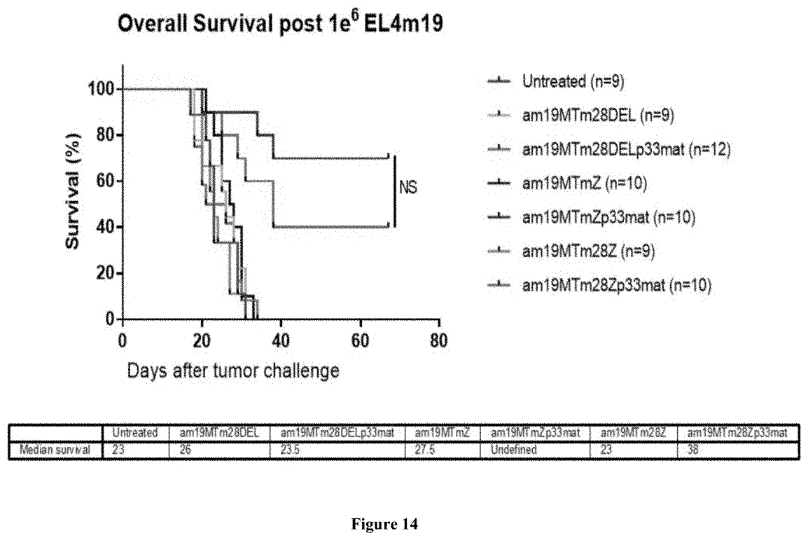

The modified T cells were next studied in the context of syngeneic, immune competent models of disease wherein long-term survival of EL4Sm19 tumor-bearing syngeneic immuno-competent mice were assessed. EL4Sm19 tumor bearing C57BL/6 mice were treated with 2.5×10⁶ modified T cells 1 day post tumor inoculation and survival was followed. As shown in FIG. 14, IL-33-secreting CAR T cells (am19MTmZp33mat and am19MTm28Zp33mat) induced long-term survival in tumor-bearing mice

### Example 3— Interleukin-33 (IL-33) Secreting CAR-T Cells in Human Model

Human IL-33 Secreting CAR Constructs

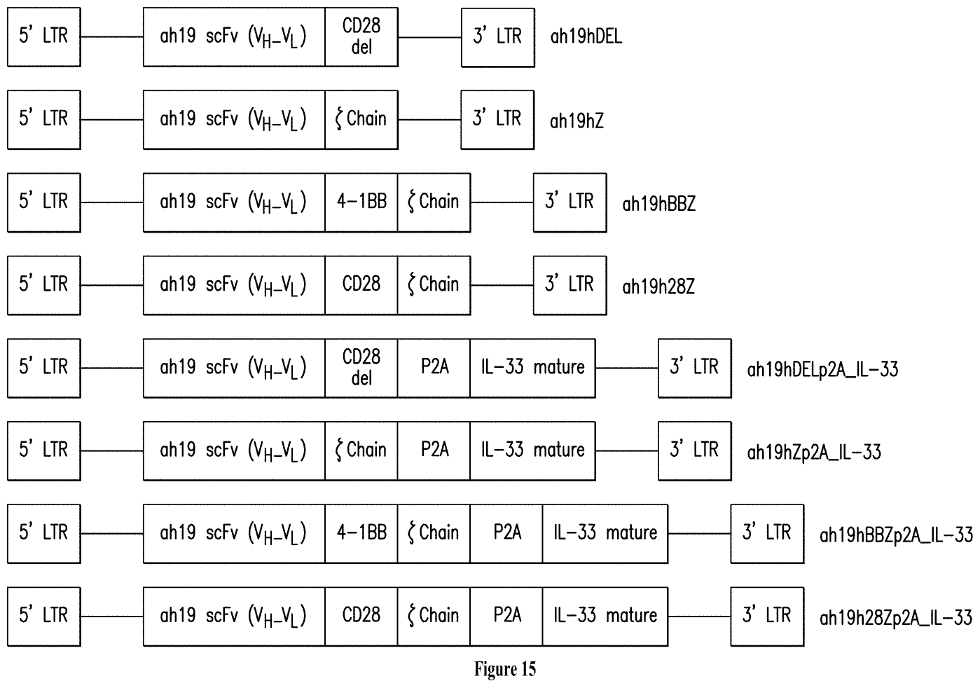

The biological effects of IL-33-secreting CAR-T cells were analyzed in a human model. Various anti-human CD19 CAR with or without a secretable human IL-33 polypeptide constructs were constructed in SFG retroviral vector backbone as shown in FIG. 15, including ah19hDEL, ah19hZ, ah19hBBZ, ah19h28Z, ah19hDELp2A_IL-33, ah19hZp2A_IL-33, ah19hBBZp2a_IL-33, and ah19h28Zp2A_IL-33. All constructs utilized a CD28 proximal extracellular and transmembrane domain as a hinge (including 4-1BB-based constructs). In all human IL-33 secreting constructs, the cytokine was separated from the CAR by a self-cleaving P2A element.

T cells were transduced with one of the above-noted constructs.

Cell Surface CAR Expression

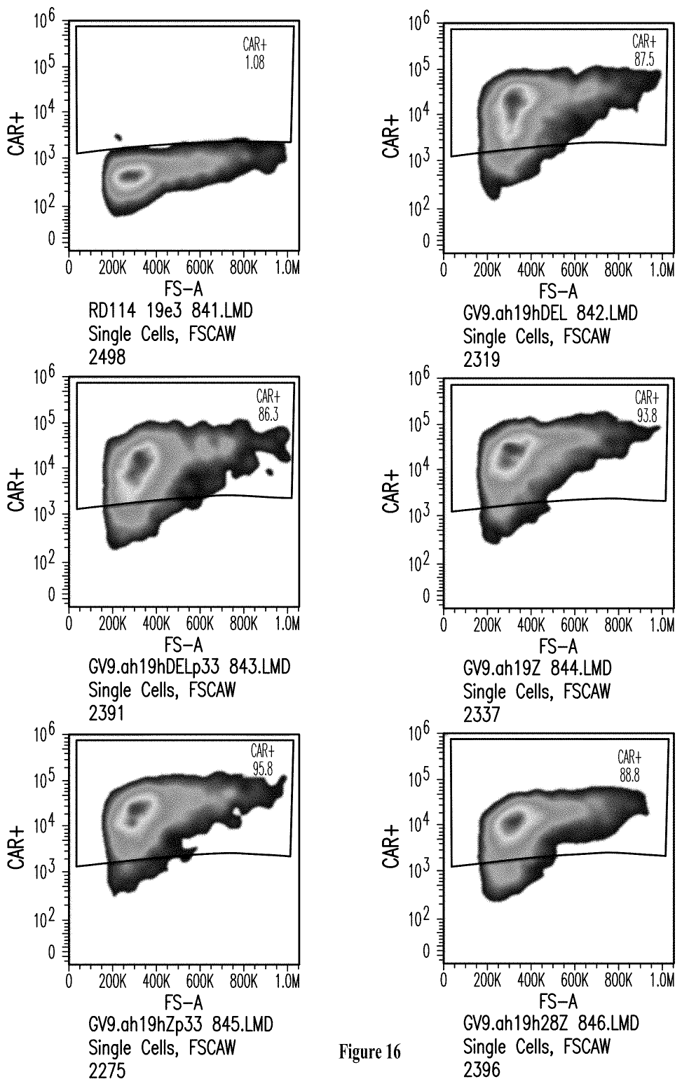

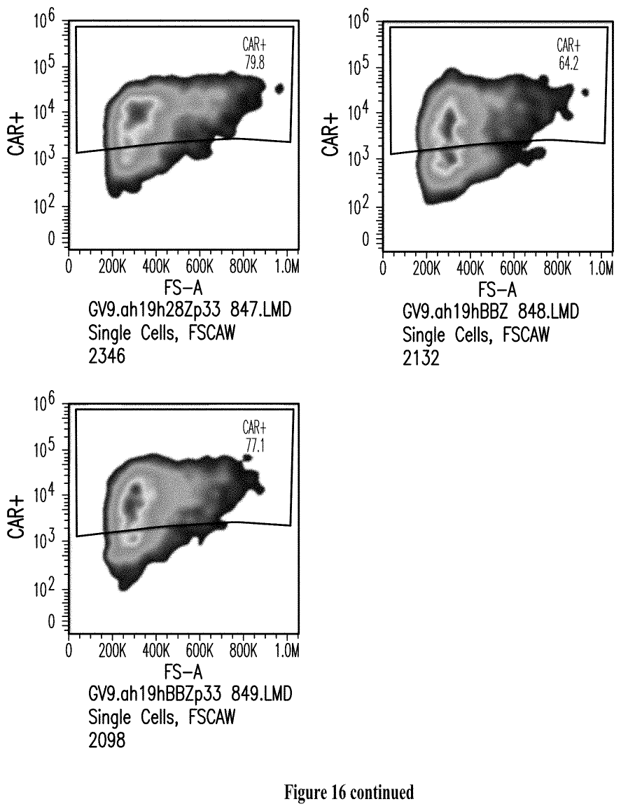

The CAR expression of the modified T cells on cell surface was analyzed by flow cytomatry and results are shown in FIG. 16. As shown in FIG. 16, GALV-pseudotyped 293GPG packaging cells stably transduced with human-based constructs had detectable CARs on their surface. Packaging cells were assessed for the presence of CAR through the use of an Alexa-647 conjugated anti-idiotype antibody specific to the mouse-derived anti-human CD19 CAR. A non-transduced RD114, a similar 293HEK cell, was used as a negative control.

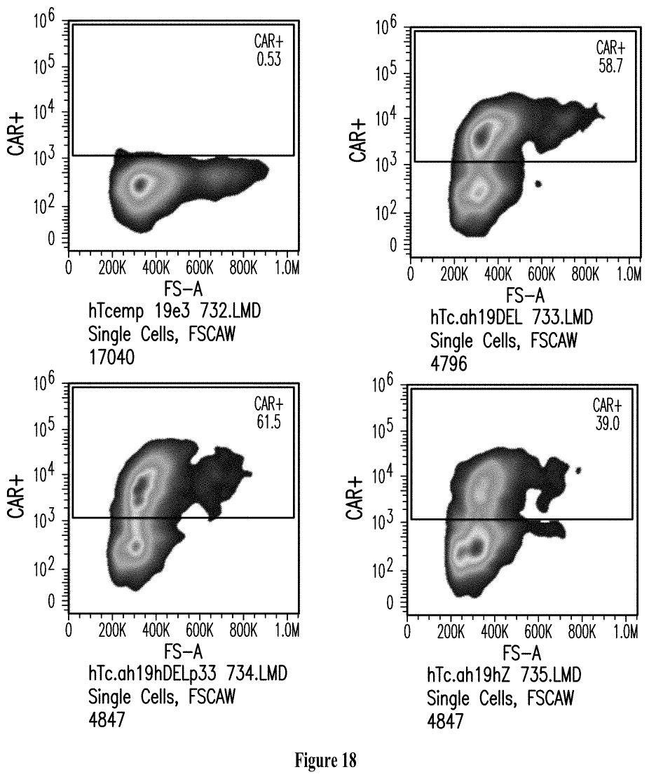

Surface CAR expression of modified T cells was also analyzed by flow cytomatry and the results are shown in FIG. 18. As shown in FIG. 18, human T cells stably transduced with human-based constructs had detectable CDRs on their surface. Human T cells were assessed for the presence of the CAR 5 days post inoculation. Non-transduced human T cells (hTcemp) served as a negative control.

Increased Cytokine Secretion

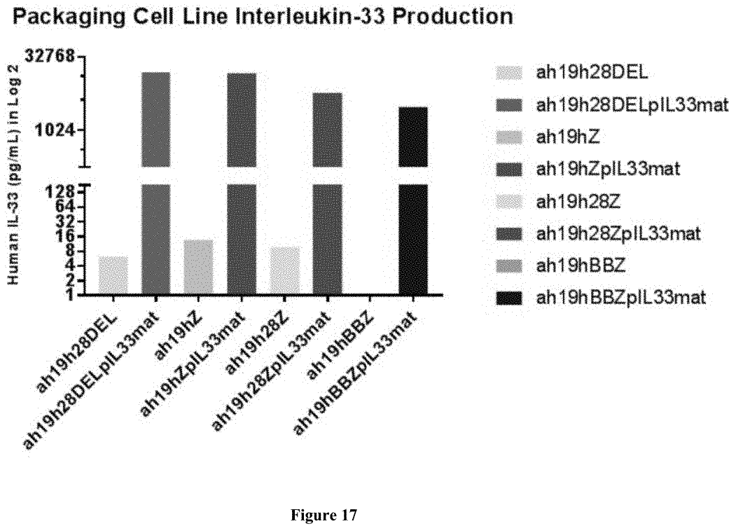

IL-33 secretion in the modified T cells was analyzed and the results are shown in FIG. 17. As shown in FIG. 17, GALV-pseudotyped 293GPG packaging cells stably transduced with human-based constructs secreted detectable levels of human IL-33. Supernatant was collected from packaging cells post retroviral transduction and assessed for cytokine concentrations by bead-based multiplex assay.

Cytolytic Efficacy

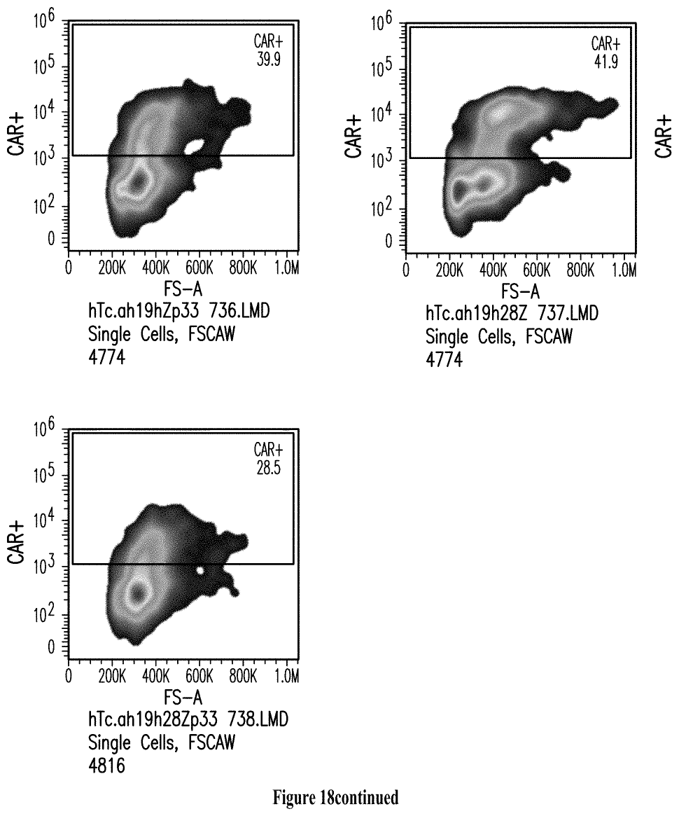

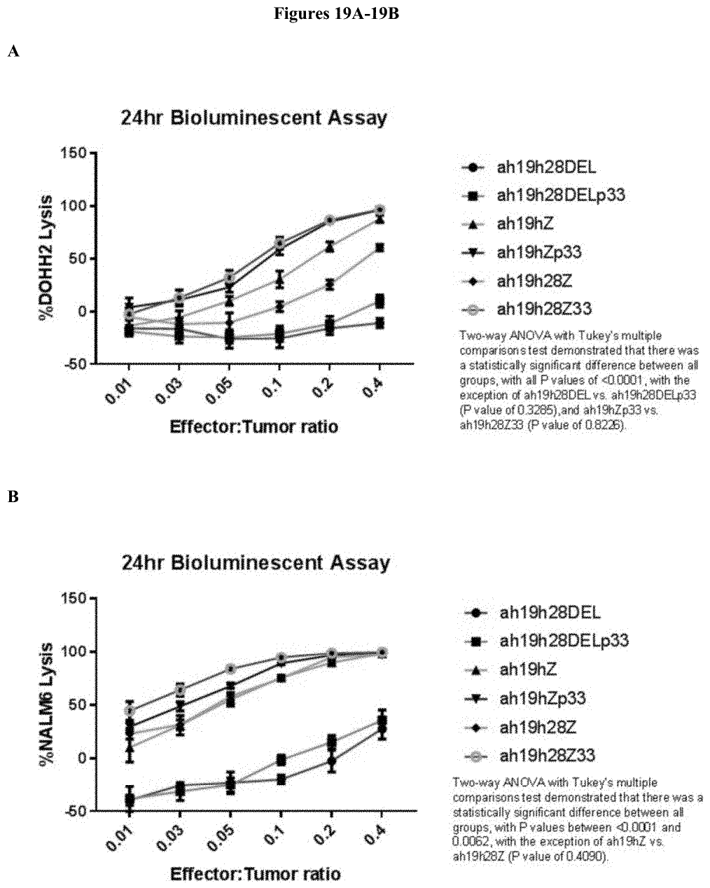

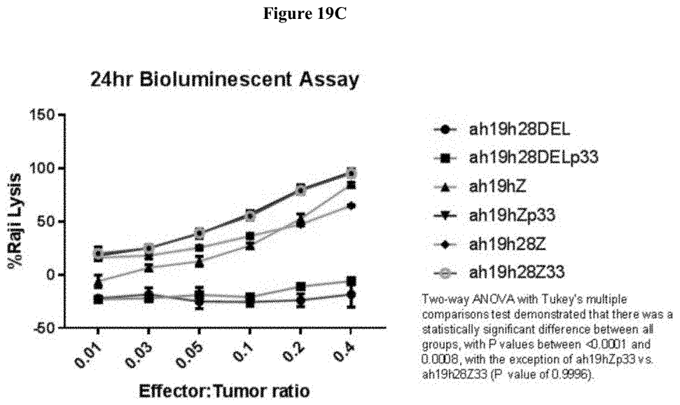

Cytolytic efficacy of the modified T cells were analyzed. CAR T cells transduced with IL-33-screting CAR constructs and control constructs were cocultured with antigen-positive tumor cells such as DOHH2, NALM6, and Raji, at different effector:tumor ratios, and tumor cell lysis was measured by bioluminescence 24 hours later. As shown in FIG. 19, IL-33-secreting human anti-human CD19 CAR-expressing T cells displayed significantly enhanced dose-dependent CD19⁺ target cell lysis compared to their non-cytokine secreting counterparts.

### Example 4

It was previously demonstrated that the combination of recombinant IL-12 and IL-33 can lead to antigen-independent secretion of interferon gamma by T cells. This phenomenon was exploited to develop an in vitro assay to determine if the IL-33 secreted by the IL33-secreting CAR T cells was functional. In this assay, addition of exogenous recombinant IL-12 to CAR T cells should lead to higher interferon gamma secretion by IL33-secreting CAR T cells over their non-IL33 secreting counterparts in the absence of antigen.

Methods and Materials

Mouse spleens were harvested and used to generate CD19-targeted CAR T cells. In this experiment, the CAR construct used incorporated a violet-excitable GFP (vG) fluorescent tag, allowing for detection of CAR T cells without the use of antibodies. Additionally, these CAR constructs also had a MYC tag incorporated after the anti-mouse CD19-targeting scFv (am19MT), allowing for CAR detection or cross-linking with anti-MYC tag antibody. Five different CAR T cell constructs were manufactured:
1. vG.am19MTdel: a non-functional CAR T cell with a truncated non-signaling CD28 intracellular domain
2. vG.am19MTZ: a first-generation CAR T cell
3. vG.am19MTZ33: a first-generation IL33-secreting CART cell
4. vG.am19MT28Z: a second-generation CD28-based CAR T cell
5. vG.am19MT28Z33: a second-generation CD28-based IL33-secreting CART cell

After manufacture, CAR T cells were subsequently seeded at 100,000 CAR-positive cells per well in a 96-well plate with/without the addition of recombinant murine IL-12 (rmIL12) for a final concentration of 10 ng/mL in triplicate. After 24 hours, supernatant was collected, and soluble cytokines were measured using a Luminex bead-based multiplex assay.

Results

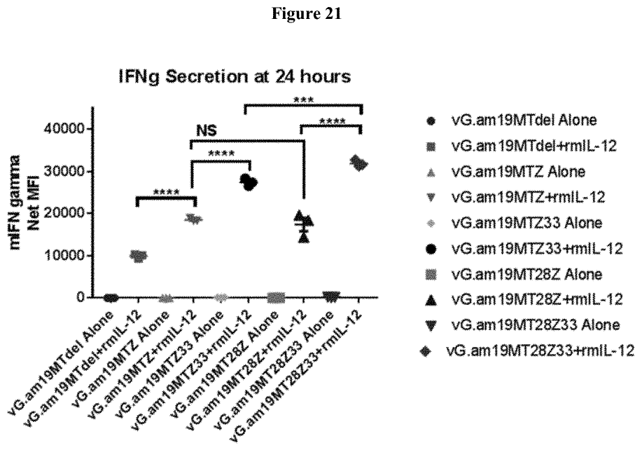

IL33-secreting constructs demonstrated increased interferon gamma secretion relative to their non-IL33 secreting counterparts in the context of 10 ng/mL rmIL12, as shown in FIG. 21. This assay demonstrated that the IL33 secreted by IL33-secreting murine CAR T cells was functional as it led to increased interferon gamma secretion by CART cells in the presence of IL-12 and the absence of antigen.

### Example 5

Similar to Example 4, the functionality of secretable IL-33 was tested in human IL33-secreting CAR T cells.

Methods and Materials

Healthy donor peripheral blood mononuclear cells (PBMCs) were utilized to generate CD19-targeted CAR T cells. In this experiment, the CAR construct used incorporated a truncated EGFR (Et), preceding the anti-human CD19-targeting scFv (ah19). Six different CAR T cells were manufactured:
1. Et.ah19hDEL: a non-functional CAR T cell with a truncated non-signaling CD28 intracellular domain
2. Et.ah19hDELp33: a non-functional CAR T cell with a truncated non-signaling CD28 intracellular domain, in line with a P2A element and a secretable IL33 gene (p33)
3. Et.ah19hZ: a first-generation CAR T cell
4. Et.ah19hZp33: a first-generation CAR T cell incorporating a P2A element and a secretable IL33 gene (p33)
5. Et.ah19h28Z: a second-generation CAR T cell
6. Et.ah19h28Zp33: a second-generation CD28-based CAR T cell incorporating a P2A element and a secretable IL33 gene (p33)

After manufacture, CAR T cells were subsequently seeded at 100,000 CAR-positive cells per well in a 96-well plate with/without the addition of recombinant human IL-12 (rhlL-12) for a final concentration of 1 and 10 ng/mL in triplicate. After 24 hours, supernatant was collected, and soluble cytokines were measured using a Luminex bead-based multiplex assay.

Results

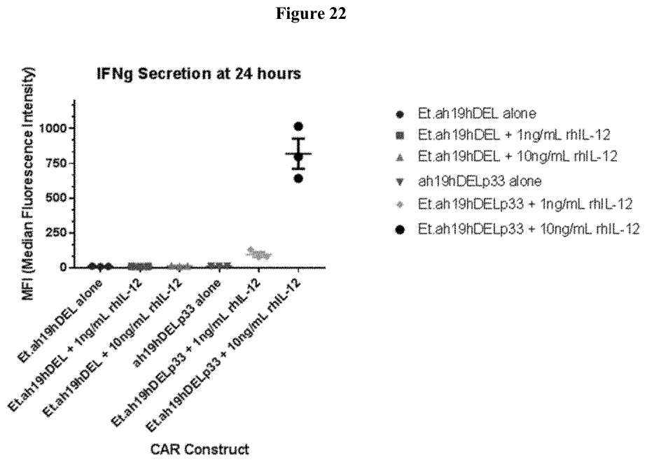

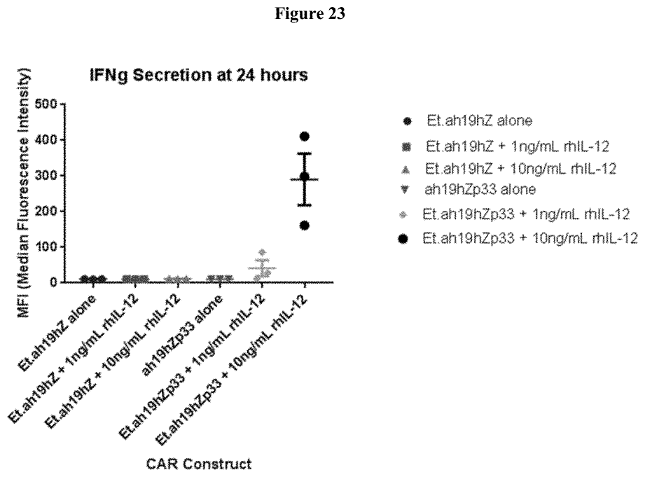

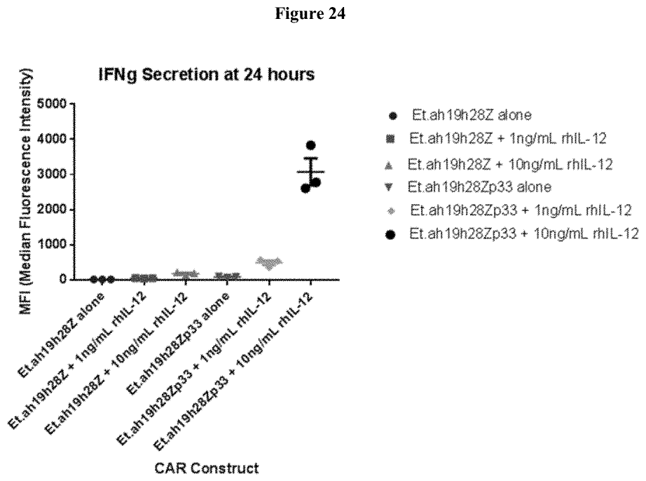

IL33-secreting constructs demonstrated increased interferon gamma secretion relative to their non-IL33 secreting counterparts in the context of increasing concentrations of rhlL-12, as shown in FIGS. 22-24. The assay demonstrated that the IL33 secreted by IL33-secreting human CAR T cells was functional as it led to increased interferon gamma secretion by CAR T cells in the presence of escalating doses of IL-12 and the absence of antigen.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of reducing tumor burden in a subject, and/or treating and/or preventing a neoplasm in a subject, and/or lengthening survival of a subject having a neoplasm, and/or increasing immune-activating cytokine production in response to a tumor antigen or a pathogen antigen in a subject, the method comprising administering to the subject an effective amount of immunoresponsive cells or a pharmaceutical composition comprising thereof, wherein the immunoresponsive comprises
(a) an antigen-recognizing receptor that binds to an antigen, and
(b) an exogenous IL-33 polypeptide or a fragment thereof.

2. The method of claim 1, wherein the exogenous IL-33 polypeptide is secreted.

3. The method of claim 1, wherein the antigen-recognizing receptor and/or the exogenous IL-33 polypeptide is expressed from a vector.

4. The method of claim 1, wherein the exogenous IL-33 polypeptide comprises a heterologous signal sequence at the amino-terminus.

5. The method of claim 4, wherein the heterologous signal sequence is selected from the group consisting of an IL-2 signal sequence, a kappa leader sequence, a CD8 leader sequence, and combinations thereof.

6. The method of claim 5, wherein the heterologous signal sequence is an IL-2 signal sequence.

7. The method of claim 1, wherein the IL-33 peptide comprises (a) an amino acid sequence that is at least about 80% homologous to the sequence set forth in SEQ ID NO: 4 or SEQ ID NO: 21, or (b) the amino acid sequence set forth in SEQ ID NO: 4 or SEQ ID NO: 21.

8. The method of claim 1, wherein the antigen is a tumor antigen or a pathogen antigen.

9. The method of claim 8, wherein the antigen is a tumor antigen.

10. The method of claim 9, wherein the tumor antigen is selected from the group consisting of CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, erb-B4, FBP, Fetal acetylcholine receptor, folate receptor-a, GD2, GD3, HER-2, hTERT, IL-13R-a2, K-light chain, KDR, LeY, L1 cell adhesion molecule, MAGE-A1, Mesothelin, ERBB2, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, and EGFR-VIII.

11. The method of claim 10, wherein the tumor antigen is CD19.

12. The method of claim 1, wherein the immunoresponsive cell is selected from the group consisting of a T cell, a Natural Killer (NK) cell, a cytotoxic T lymphocyte (CTL), a regulatory T cell, a Natural Killer T (NKT) cell, and a pluripotent stem cell from which lymphoid cells may be differentiated.

13. The method of claim 12, wherein the immunoresponsive cell is a T cell.

14. The method of claim 13, wherein the T cell is selected from the group consisting of a cytotoxic T lymphocyte (CTL), a regulatory T cell, a Natural Killer T (NKT) cell, and combinations thereof.

15. The method of claim 1, wherein the antigen-recognizing receptor is a T cell receptor (TCR) or a chimeric antigen receptor (CAR).

16. The method of claim 15, wherein the antigen-recognizing receptor is a CAR.

17. The method of claim 16, wherein the CAR comprises an extracellular antigen-binding domain, a transmembrane domain, and an intracellular signaling domain.

18. The method of claim 17, wherein the intracellular signaling of the CAR does not comprise a co-stimulatory signaling region.

19. The method of claim 1, wherein the exogenous IL-33 polypeptide enhances an immune response of the immunoresponsive cell, and/or increases anti-tumor cytokine production of the immunoresponsive cell.

20. The method of claim 19, wherein the anti-tumor cytokine is selected from the group consisting of IL-2, GM-CSF and IFN-γ.

21. The method of claim 1, wherein the subject did not receive preconditioning chemotherapy prior to the administration of the cells.

22. A method of reducing tumor burden in a subject, and/or treating and/or preventing a neoplasm in a subject, and/or lengthening survival of a subject having a neoplasm, and/or increasing immune-activating cytokine production in response to a tumor antigen or a pathogen antigen in a subject, the method comprising administering to the subject an effective amount of immunoresponsive cells or a pharmaceutical composition comprising thereof, wherein the immunoresponsive comprises:
(a) an antigen-recognizing receptor that binds to an antigen, and
(b) a modified promoter at an endogenous IL-33 gene locus, wherein the modified promoter enhances gene expression of the endogenous IL-33 gene.

23. The method of claim 22, wherein the modification comprises replacement of an endogenous promoter with a constitutive promoter or an inducible promoter, or insertion of a constitutive promoter or inducible promoter to the promoter region of the endogenous IL-33 gene locus.

24. The method of claim 23, wherein the constitutive promoter is selected from the group consisting of a CMV promoter, an EF1a promoter, a SV40 promoter, a PGK1 promoter, a Ubc promoter, a beta-actin promoter, and a CAG promoter.

25. The method of claim 23, wherein the inducible promoter is selected from the group consisting of a tetracycline response element (TRE) promoter and an estrogen response element (ERE) promoter.
